(19) United States
(12) Patent Application Publication
(10) Pub. No.: US 2025/0090497 A1
(43) Pub. Date: Mar. 20, 2025

(54) METHODS OF TREATING CONDITIONS RELATED TO THE S1P1 RECEPTOR

(71) Applicant: Arena Pharmaceuticals, Inc., New York, NY (US)

(72) Inventors: Ronald J. Christopher, Carlsbad, CA (US); Abu J.M. Sadeque, San Diego, CA (US)

(73) Assignee: Arena Pharmaceuticals, Inc., New York, NY (US)

(21) Appl. No.: 18/964,847

(22) Filed: Dec. 2, 2024

Related U.S. Application Data
(63) Continuation of application No. 15/734,920, filed on Dec. 3, 2020, now Pat. No. 12,156,866.
(60) Provisional application No. 62/681,426, filed on Jun. 6, 2018.
(60) Provisional application No. 62/746,946, filed on Oct. 17, 2018.
(60) Provisional application No. 62/850,470, filed on May 20, 2019.

(51) Int. Cl. A61K 31/403; A61K 45/06

(52) U.S. Cl. CPC A61K 31/403; A61K 45/06

(57) ABSTRACT

Provided are methods of treatment of a sphingosine 1-phosphate subtype 1 (S1P₁) receptor-associated disorder comprising prescribing and/or administering to an individual in need thereof a standard dose of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof.

### FIELD

Provided are methods useful in the treatment of sphingosine 1-phosphate subtype 1 (S1P₁ or SIP1) receptor-associated disorders.

The sphingosine-1-phosphate (S1P) receptors 1-5 constitute a family of G protein-coupled receptors with a seven-transmembrane domain. These receptors, referred to as S1P₁ to S1P₅ (formerly termed endothelial differentiation gene (EDG) receptor-1, -5, -3, -6, and -8, respectively; Chun et al., Pharmacological Reviews, 54:265-269, 2002), are activated via binding by sphingosine-1-phosphate, which is produced by the sphingosine kinase-catalyzed phosphorylation of sphingosine. S1P₁, S1P₄, and S1P₅ receptors activate Gi but not Gq, whereas S1P₂ and S1P₃ receptors activate both Gi and Gq. The S1P₃ receptor, but not the S1P₁ receptor, responds to an agonist with an increase in intracellular calcium.

In view of the growing demand for S1P₁ agonists useful in the treatment of S1P₁ receptor-associated disorders, the compound (R)-2-(7-(4-cyclopentyl-3-(trifluoromethyl)benzyloxy)-1,2,3,4-tetrahydrocyclopenta[b]indol-3-yl)acetic acid (Compound 1, APD334), or a pharmaceutically acceptable salt, solvate, or hydrate thereof,

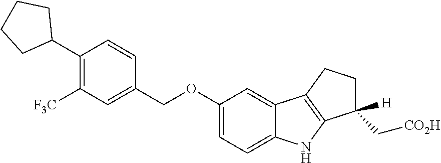 [SMILES: O=C(O)C[C@H]1CCc2c1[nH]c1ccc(OCc3ccc(C4CCCC4)c(C(F)(F)F)c3)cc21]

has emerged as an important new compound, see PCT patent application, Serial No. PCT/US2009/004265 hereby incorporated by reference in its entirety. Compound 1, or a pharmaceutically acceptable salt, solvate, or hydrate thereof, is an investigational drug candidate intended for the treatment of sphingosine 1-phosphate subtype 1 (S1P₁) receptor-associated disorders.

Many S1P₁ agonists cause side effects, and particularly cardiovascular related adverse events, that require that doctors titrate patients slowly to a maintenance dose. This titration period can take weeks or even a month. The complexity and length of the titration regimen may result in prematurely discontinuing therapy by patients prior to reaching the maintenance dose or to doctors preferring other therapeutic options.

There exists a need for effectively treating individuals who are in need of treatment with Compound 1, or a pharmaceutically acceptable salt, solvate, or hydrate thereof. The present disclosure satisfies this need and provides related advantages as well.

Citation of any reference throughout this application is not to be construed as an admission that such reference is prior art to the present application.

### SUMMARY

Provided is a method of treating an individual with a sphingosine 1-phosphate subtype 1 (S1P₁) receptor-associated disorder comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, wherein the pharmaceutical dosage form has a mean fed/fasted ratio of the area under the plasma concentration versus time curve of from about 0.8 to about 1.25 and a mean fed/fasted ratio of the maximum plasma concentration (Cmax) from about 0.8 to about 1.25.

Also provided is a method of administering a sphingosine 1-phosphate subtype 1 (S1P₁) receptor modulator chosen from (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to a patient in need thereof wherein the patient is also being administered a cytochrome P450 2C8 (CYP2C8) inhibitor, cytochrome P450 2C9 (CYP2C9) inhibitor, UDP-glucuronosyltransferase (UGT) enzyme UGT1A1 inhibitor, or UDP-glucuronosyltransferase (UGT) enzyme UGT1A6 inhibitor, comprising: administering to the patient a therapeutically effective amount of the S1P₁ modulator, wherein the therapeutically effective amount of the S1P₁ modulator is less than the amount that would be administered to a patient who is not also being administered a CYP2C8 inhibitor, CYP2C9 inhibitor, UGT1A1 inhibitor, or UGT1A6 inhibitor.

Also provided is a method of administering a sphingosine 1-phosphate subtype 1 (S1P₁) receptor modulator chosen from (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to a patient in need thereof, comprising: administering to the patient a therapeutically effective amount of the S1P₁ modulator, subsequently determining that the patient is to begin treatment with a CYP2C8 inhibitor, CYP2C9 inhibitor, UDP-glucuronosyltransferase (UGT) enzyme UGT1A1 inhibitor, or UDP-glucuronosyltransferase (UGT) enzyme UGT1A6 inhibitor and administering the S1P₁ modulator in an amount that is less than the amount that would be administered to a patient who is not also being administered CYP2C8 inhibitor, CYP2C9 inhibitor, UDP-glucuronosyltransferase (UGT) enzyme UGT1A1 inhibitor, or UDP-glucuronosyltransferase (UGT) enzyme UGT1A6 inhibitor.

Also provided is a method of administering a sphingosine 1-phosphate subtype 1 (S1P₁) receptor modulator chosen from (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to a patient in need thereof wherein the patient is also being administered a substrate of a membrane transporter wherein the membrane transporter is selected from P-glycoprotein (Pgp), BCRP (breast cancer resistance protein), and OATP1B1, comprising: administering to the patient a therapeutically effective amount of the S1P₁ modulator.

Also provided is a method of administering a sphingosine 1-phosphate subtype 1 (S1P₁) receptor modulator chosen from R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt and/or isotopic variant thereof, to a patient in need thereof, comprising: administering to the patient a therapeutically effective amount of the S1P₁ receptor modulator, subsequently determining that the patient is to begin treatment with a substrate of a membrane transporter wherein the membrane transporter is selected from P-glycoprotein (Pgp), BCRP (breast cancer resistance protein), and OATP1B1, and continuing administration of the therapeutically effective amount of the S1P₁ receptor modulator to the patient.

These and other aspects of the invention disclosed herein will be set forth in greater detail as the patent disclosure proceeds.

### BRIEF DESCRIPTION OF THE DRAWINGS

FIG. 1 shows the mean plasma concentration-time profiles for the tablet formulation of Compound 1 administered under fed versus fasted conditions.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)-2-(7-(4-cyclopentyl-3-(trifluoromethyl)benzyloxy)-1,2,3,4-tetrahydrocyclopenta[b]indol-3-yl)acetic acid including crystalline forms thereof. As a non-limiting example, Compound 1 may be present as an anhydrous, non-solvated crystalline form as described in WO 2010/011316 (incorporated by reference herein in its entirety). As another non-limiting example, an L-arginine salt of Compound 1 may be present as an anhydrous, non-solvated crystalline form as described in WO 2010/011316 and WO 2011/094008 (each of which is incorporated by reference herein in its entirety). As another non-limiting example, a calcium salt of Compound 1 may be present as a crystalline form as described in WO 2010/011316 (incorporated by reference herein in its entirety).

ADMINISTERING: As used herein, “administering” means to provide a compound or other therapy, remedy, or treatment such that an individual internalizes a compound.

CO-ADMINISTER: As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a sphingosine 1-phosphate subtype 1 (S1P₁) receptor-associated disorder. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention” such as prevention of a sphingosine 1-phosphate subtype 1 (S1P₁) receptor-associated disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

ADVERSE EVENT: As used herein, an “adverse event” is an untoward medical occurrence that is associated with treatment with Compound 1 or a pharmaceutically acceptable salt, solvate, or hydrate thereof. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is an abnormal liver function test, such as an elevated ALT & AST>2×ULN. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDIVIDUAL: As used herein, “individual” means any animal, including mammals, preferably mice, rats, other rodents, rabbits, dogs, cats, swine, cattle, sheep, horses, or primates and most preferably humans. In some embodiments, a human individual is referred to a “patient.”

ACUTE HEART RATE REDUCTION: As used herein, “acute heart rate reduction” means a heart rate decrease from normal sinus rhythm of, for example, 10 or more beats per minute (bpm), such as less than about 5 bpm, e.g., less than about 4 bpm or less than about 3 bpm or less than 2 bpm, that is maximal within a few hours, for example 1-3 hours, after drug administration, and thereafter the heart rate returns towards the pre-dose value.

NORMAL SINUS RHYTHM: As used herein, “normal sinus rhythm” means the sinus rhythm of the individual when not undergoing treatment. The evaluation of normal sinus rhythm is within the ability of a physician. A normal sinus rhythm will generally give rise to a heart rate in the range from 60-100 bpm.

DOSE: As used herein, “dose” means a quantity of Compound 1, or a pharmaceutically acceptable salt, solvate, or hydrate thereof, given to the individual for treating or preventing the disease or disorder at one specific time.

STANDARD DOSE: As used herein, “standard dose” means the dose of Compound 1, or a pharmaceutically acceptable salt, solvate, or hydrate thereof, that is given to the individual for treating or preventing the disease or disorder. In some embodiments, administration of the standard dose achieves a target reduction in peripheral blood lymphocyte counts, e.g., a reduction in baseline of at least 35%, such as at least 40%, such as at least 45%, such as at least 50%, such as at least 55%, such as at least 60%, such as at least 65%, such as at least 70%. In some embodiments, administration of the standard dose achieves a reduction in baseline of about 35% to about 70%, such as about 40% to about 65%, such as about 50% to about 65%. In some embodiments, administration of the standard dose achieves target peripheral blood lymphocyte counts, e.g., less than 1000 lymphocytes per microliter, such as 400-800 lymphocytes per microliter. The target dose may vary depending on the nature and severity of the disease to be treated.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

FASTED INDIVIDUAL: As used herein, “fasted individual” means an individual who has not eaten any food, i.e., has fasted for at least 6-8 hours, such as about 8 hours, before the administration of Compound 1, or a pharmaceutically acceptable salt, hydrate, or solvate thereof, and who does not eat any food and continues to fast for at least 1 hour after the administration of Compound 1, or a pharmaceutically acceptable salt, hydrate, or solvate thereof. In certain embodiments, the individual may also refrain from ingesting certain non-food substances during the fasting period. For example, in certain embodiments the individual does not ingest any supplements and/or drugs during the fasting period. In certain embodiments, the individual does not ingest any high calorie liquids during the fasting period. In certain embodiments, the individual does not ingest any liquids other than water during the fasting period. In certain embodiments, the individual may ingest small amounts of low-calorie beverages, such as tea, coffee, or diluted juices.

MAYO CLINIC SCORE (MCS): As used herein, “Mayo Clinic Score” or “MCS” means an instrument designed to measure disease activity of ulcerative colitis and consists of up to 4 subscores: stool frequency, rectal bleeding, findings of flexible proctosigmoidoscopy, and physician global assessment with each component ranging from 0 to 3 (0=normal, 1=mild, 2=moderate, 3=severe). Total score therefore ranges from 0 to 12, with a higher score indicating more severe disease. The 6-point Mayo score is based on stool frequency and rectal bleeding PROs collected daily using electronic patient diaries and excludes the findings on endoscopy and the physician's global assessment. The 3-point Mayo score is based on stool frequency, rectal bleeding, and findings on endoscopy and has a total score ranging from 0 to 9. The 2-point Mayo score is based on rectal bleeding and findings on endoscopy and has a total score ranging from 0 to 6. The physician's global assessment acknowledges the three other criteria findings of the MCS, the individual's daily record of abdominal discomfort and general sense of well-being, and other observations, such as physical findings and the individual's performance.

MILDLY TO MODERATELY ACTIVE ULCERATIVE COLITIS: As used herein, “mildly to moderately active ulcerative colitis” means ulcerative colitis characterized by a 4-component MCS of 4 to 10.

MODERATELY TO SEVERELY ACTIVE ULCERATIVE COLITIS: As used herein, “moderately to severely active ulcerative colitis” means ulcerative colitis characterized by a 3-component MCS of 4 to 9 including an endoscopic subscore of ≥2 and a rectal bleeding score of ≥1. The 3-component MCS uses 3 of the 4 components of the complete MCS (endoscopic findings, rectal bleeding, and stool frequency).

CLINICAL REMISSION: As used herein, “clinical remission” with respect to ulcerative colitis means a 3-component Mayo Clinic score as follows: an endoscopy score (using flexible proctosigmoidoscopy) of 0 or 1, a rectal bleeding score of 0, and a stool frequency score of 0 or 1 with a decrease of ≥1 point from baseline subscore.

CLINICAL RESPONSE: As used herein, “clinical response” with respect to ulcerative colitis means a reduction in the 3-component Mayo Clinic score of ≥2 points and a decrease of ≥30% from baseline with an accompanying decrease in rectal bleeding subscore of ≥1 or absolute rectal bleeding score of 0 or 1.

ENDOSCOPIC IMPROVEMENT: As used herein, “endoscopic improvement” with respect to ulcerative colitis means ulcerative colitis characterized by a Mayo endoscopic subscore (using findings of flexible proctosigmoidoscopy) of ≤1 point.

ENDOSCOPIC REMISSION: As used herein, “endoscopic remission” with respect to ulcerative colitis means ulcerative colitis characterized by findings from flexible proctosigmoidoscopy subscore of the Mayo Clinic score=0.

IMPROVEMENT IN RECTAL BLEEDING: As used herein, “improvement in rectal bleeding” with respect to ulcerative colitis means a change from baseline <0.

HISTOLOGIC HEALING/HISTOLOGIC IMPROVEMENT: As used herein, “histologic healing,” “histological healing,” “histologic improvement,” or “histological improvement” with respect to ulcerative colitis means a score of <3.1 on the Geboes Index.

HISTOLOGIC REMISSION: As used herein, “histologic remission” or “histological remission” with respect to ulcerative colitis means a score of <2.0 on the Geboes Index.

MUCOSAL HEALING: As used herein, “mucosal healing” is both endoscopic improvement and histological remission.

IMPROVEMENT IN STOOL FREQUENCY: As used herein, “improvement in stool frequency” with respect to ulcerative colitis means a change from baseline <0.

5-AMINOSALICYLATES: As used herein, “5-aminosalicylates”, means a class of drugs that include, for example, CANASA® (mesalamine), COLAZAL® (balsalazide disodium), ASACOL® (mesalamine), DELZICOL® (mesalamine), and DIPENTUM® (olsalazine).

IMMUNOSUPPRESSIVES: As used herein, “immunosuppressives”, means a class of drugs that include, for example, AZASAN® (azathioprine), IMURAN® (azathioprine), GENGRAF® (cyclosporine), NEORAL® (cyclosporine), and SANDIMMUNE® (cyclosporine).

GLUCOCORTICOSTEROIDS: As used herein, “glucocorticosteroids”, means a class of drugs that include, for example, UCERIS® (budesonide); DELTASONE® (prednisone), MEDROL® (methylprednisolone), and hydrocortisone.

TNFα ANTAGONISTS: As used herein, “TNFα antagonists” or “tumor necrosis factor-α antagonists”, means a class of drugs that include, for example, SIMPONI® (golimumab), REMICADE® (infliximab), HUMIRA® (adalimumab), and CIMZIA® (certolizumab pegol).

INTEGRIN RECEPTOR ANTAGONISTS: As used herein, “integrin receptor antagonists” means a class of drugs that include, for example, ENTYVIO® (vedolizumab).

PHARMACEUTICAL COMPOSITION: As used here, “pharmaceutical composition” means a composition comprising at least one active ingredient, such as Compound 1; including but not limited to, salts, solvates, and hydrates of Compound 1, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

AGONIST: As used herein, “agonist” means a moiety that interacts with and activates a G-protein-coupled receptor, such as the S1P₁ receptor, such as can thereby initiate a physiological or pharmacological response characteristic of that receptor. For example, an agonist activates an intracellular response upon binding to the receptor or enhances GTP binding to a membrane. In certain embodiments, an agonist of the invention is an S1P₁ receptor agonist that is capable of facilitating sustained S1P₁ receptor internalization (see e.g., Matloubian et al., Nature, 427, 355, 2004).

ANTAGONIST: As used herein, “antagonist” means a moiety that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

INVERSE AGONIST: As used herein, “inverse agonist” means a moiety that binds to the endogenous form of the receptor or to the constitutively activated form of the receptor and which inhibits the baseline intracellular response initiated by the active form of the receptor below the normal base level of activity which is observed in the absence of an agonist or partial agonist, or decreases GTP binding to a membrane. In some embodiments, the baseline intracellular response is inhibited in the presence of the inverse agonist by at least 30%. In some embodiments, the baseline intracellular response is inhibited in the presence of the inverse agonist by at least 50%. In some embodiments, the baseline intracellular response is inhibited in the presence of the inverse agonist by at least 750%, as compared with the baseline response in the absence of the inverse agonist.

HYDRATE: As used herein, “hydrate” means a compound of the invention or a salt thereof, that further includes a stoichiometric or non-stoichiometric amount of water bound by non-covalent intermolecular forces.

SAFETY POPULATION: As used herein, “safety population” means all randomized subjects who received study medication.

SOLVATE: As used herein, “solvate” means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It is understood that when the phrase “pharmaceutically acceptable salts, solvates and hydrates” or the phrase “pharmaceutically acceptable salt, solvate, or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts.

It will be apparent to those skilled in the art that the dosage forms described herein may comprise, as the active component, either Compound 1 or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, DE), Avantium Technologies (Amsterdam) and Aptuit (Greenwich, CT).

The present disclosure includes all isotopes of atoms occurring in the present compounds, salts, solvates, and hydrates. Isotopes include those atoms having the same atomic number but different mass numbers. One aspect of the present invention includes every combination of one or more atoms in the present compounds, salts, solvates, and hydrates that is replaced with an atom having the same atomic number but a different mass number. One such example is the replacement of an atom that is the most naturally abundant isotope, such as ¹H or ¹²C, found in one the present compounds, salts, solvates, and hydrates, with a different atom that is not the most naturally abundant isotope, such as ²H or ³H (replacing ¹H), or ¹¹C, ¹³C, or ¹⁴C (replacing ¹²C). When such a replacement has taken place, it is commonly referred to as being isotopically labeled. Isotopic-labeling of the present compounds, salts, solvates, and hydrates can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸O. An isotope of fluorine includes ¹⁸F. An isotope of sulfur includes ³¹S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present compounds, salts, solvates, and hydrates, wherein the naturally occurring distribution of the isotopes in the composition is perturbed. Another aspect of the present invention includes compositions and pharmaceutical compositions comprising the compounds, salts, solvates, and hydrates, as described herein wherein the salt is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

Compounds of the present invention can be converted to “prodrugs.” The term “prodrugs” means compounds that have been modified with specific chemical groups known in the art and that when administered into an individual undergo biotransformation to give the parent compound. Prodrugs can thus be viewed as compounds of the invention containing one or more specialized non-toxic protective groups used in a transient manner to alter or to eliminate a property of the compound. In one general aspect, the “prodrug” approach is utilized to facilitate oral absorption. A thorough discussion is provided in T. Higuchi and V. Stella, Prodrugs as Novel Delivery Systems Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

AUC: As used herein, “AUC” refers to the area under the curve, or the integral, of the plasma concentration of an active pharmaceutical ingredient or metabolite over time following a dosing event.

AUC₀₋ₜ: As used herein “AUC₀₋₄” is the integral under the plasma concentration curve from time 0 (dosing) to time “t”.

AUC_{0-∞}: As used herein, “AUC₀₋₂₈.” is the AUC from time 0 (dosing) to time infinity. Unless otherwise stated, AUC refers to AUC_{0-∞}.

Cmax: As used herein, Cmax (or Cₘₐₓ) is a pharmacokinetic parameter denoting the maximum observed blood plasma concentration following delivery of an active pharmaceutical ingredient. Cmax occurs at the time of maximum plasma concentration, tₘₐₓ.

tₘₐₓ: As used herein, “tₘₐₓ” is a pharmacokinetic parameter denoting the time to maximum blood plasma concentration following delivery of an active pharmaceutical ingredient

t_{1/2}: As used herein, “t_{1/2}” or “plasma half-life” or “elimination half-life” or the like is a pharmacokinetic parameter denoting the apparent plasma terminal phase half-life, i.e., the time, after absorption and distribution of a drug is complete, for the plasma concentration to fall by half.

As used herein, “a substance having a narrow therapeutic index” means a substance falling within any definition of narrow therapeutic index as promulgated by the U.S. Food and Drug Administration or any successor agency thereof, for example, a substance having a less than 2-fold difference in median lethal dose (LD50) and median effective dose (ED50) values or having a less than 2-fold difference in the minimum toxic concentration and minimum effective concentration in the blood; and for which safe and effective use of the substance requires careful titration and patient monitoring.

As used herein, a substance is a “substrate” of enzyme activity when it can be chemically transformed by action of the enzyme on the substance. “Enzyme activity” refers broadly to the specific activity of the enzyme (i.e., the rate at which the enzyme transforms a substrate per mg or mole of enzyme) as well as the metabolic effect of such transformations. Thus, a substance is an “inhibitor” of enzyme activity when the specific activity or the metabolic effect of the specific activity of the enzyme can be decreased by the presence of the substance, without reference to the precise mechanism of such decrease. For example, a substance can be an inhibitor of enzyme activity by competitive, non-competitive, allosteric or other type of enzyme inhibition, by decreasing expression of the enzyme, or other direct or indirect mechanisms. Similarly, a substance is an “inducer” of enzyme activity when the specific activity or the metabolic effect of the specific activity of the enzyme can be increased by the presence of the substance, without reference to the precise mechanism of such increase. For example, a substance can be an inducer of enzyme activity by increasing reaction rate, by increasing expression of the enzyme, by allosteric activation or other direct or indirect mechanisms. Any of these effects on enzyme activity can occur at a given concentration of active agent in a single sample, donor, or patient without regard to clinical significance. It is possible for a substance to be a substrate, inhibitor, or inducer of an enzyme activity. For example, the substance can be an inhibitor of enzyme activity by one mechanism and an inducer of enzyme activity by another mechanism. The function (substrate, inhibitor, or inducer) of the substance with respect to activity of an enzyme can depend on environmental conditions. Lists of inhibitors, inducers and substrates for CYP3A4 can be found, for instance, at http://www.genemedrx.com/Cytochrome_P450_Metabolism_Table.php, and other sites.

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e. one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1 or a pharmaceutically acceptable salt, solvate, or hydrate thereof can be separated into two methods; one method reciting prescribing Compound 1 or a pharmaceutically acceptable salt, solvate, or hydrate thereof and the other method reciting administering Compound 1 or a pharmaceutically acceptable salt, solvate, or hydrate thereof. In addition, for example, a method that recites prescribing Compound 1 or a pharmaceutically acceptable salt, solvate, or hydrate thereof and a separate method of the invention reciting administering Compound 1 or a pharmaceutically acceptable salt, solvate, or hydrate thereof can be combined into a single method reciting prescribing and/or administering Compound 1 or a pharmaceutically acceptable salt, solvate, or hydrate thereof.

Provided herein are methods of co-administering Compound 1 with another drug. Also provided herein are methods of reducing the dosage of Compound 1 when co-administered with another drug. Also provided herein are methods of reducing the dosage of another drug when co-administered with Compound 1. Also provided herein are methods of discontinuing the administration of Compound 1 when an individual is administered another drug. Also provided herein are methods of discontinuing the administration of another drug when an individual is administered Compound 1. Also provided herein are methods of continuing the administration of Compound 1 when an individual is administered another drug.

Also provided herein are methods of continuing the administration of another drug when an individual is administered Compound 1. Also provided herein are methods of monitoring an individual who is co-administered Compound 1 and another drug. Also provided herein are methods of titrating the dosage of Compound 1 when co-administered with another drug. Also provided herein are methods of titrating the dosage of another drug when co-administered with Compound 1. Also provided herein are methods that entail combinations of the foregoing methods.

Provided is a method of treating an individual with a sphingosine 1-phosphate subtype 1 (S1P₁) receptor-associated disorder comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, wherein the pharmaceutical dosage form has a mean fed/fasted ratio of the area under the plasma concentration versus time curve of from about 0.8 to about 1.25 and a mean fed/fasted ratio of the maximum plasma concentration (Cmax) from about 0.8 to about 1.25.

Also provided is a method of administering a sphingosine 1-phosphate subtype 1 (S1P₁) receptor modulator chosen from (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to a patient in need thereof wherein the patient is also being administered a cytochrome P450 2C8 (CYP2C8) inhibitor, cytochrome P450 2C9 (CYP2C9) inhibitor, UDP-glucuronosyltransferase (UGT) enzyme UGT1A1 inhibitor, or UDP-glucuronosyltransferase (UGT) enzyme UGT1A6 inhibitor, comprising: administering to the patient a therapeutically effective amount of the S1P₁ modulator, wherein the therapeutically effective amount of the S1P₁ modulator is less than the amount that would be administered to a patient who is not also being administered a CYP2C8 inhibitor, CYP2C9 inhibitor, UGT1A1 inhibitor, or UGT1A6 inhibitor.

Also provided is a method of administering a sphingosine 1-phosphate subtype 1 (S1P₁) receptor modulator chosen from (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to a patient in need thereof, comprising: administering to the patient a therapeutically effective amount of the S1P₁ modulator, subsequently determining that the patient is to begin treatment with a CYP2C8 inhibitor, CYP2C9 inhibitor, UDP-glucuronosyltransferase (UGT) enzyme UGT1A1 inhibitor, or UDP-glucuronosyltransferase (UGT) enzyme UGT1A6 inhibitor and administering the S1P₁ modulator in an amount that is less than the amount that would be administered to a patient who is not also being administered CYP2C8 inhibitor, CYP2C9 inhibitor, UDP-glucuronosyltransferase (UGT) enzyme UGT1A1 inhibitor, or UDP-glucuronosyltransferase (UGT) enzyme UGT1A6 inhibitor.

In some embodiments, the method further comprises informing the patient or a medical care worker that administration of the S1P₁ modulator to a patient who is also being administered a CYP2C8 inhibitor, CYP2C9 inhibitor, UDP-glucuronosyltransferase (UGT) enzyme UGT1A1 inhibitor, or UDP-glucuronosyltransferase (UGT) enzyme UGT1A6 inhibitor results in higher exposure of the S1P₁ modulator than administration of the S1P₁ modulator to a patient who is not being administered a CYP2C8 inhibitor, CYP2C9 inhibitor, UDP-glucuronosyltransferase (UGT) enzyme UGT1A1 inhibitor, or UDP-glucuronosyltransferase (UGT) enzyme UGT1A6 inhibitor.

In some embodiments, the method further comprises informing the patient or a medical care worker that administration of the S1P₁ modulator to a patient who is also being administered a CYP2C8 inhibitor, CYP2C9 inhibitor, UDP-glucuronosyltransferase (UGT) enzyme UGT1A1 inhibitor, or UDP-glucuronosyltransferase (UGT) enzyme UGT1A6 inhibitor may result in increased risk of one or more exposure-related adverse reactions than administration of the S1P₁ modulator to a patient who is not being administered a CYP2C8 inhibitor, CYP2C9 inhibitor, UDP-glucuronosyltransferase (UGT) enzyme UGT1A1 inhibitor, or UDP-glucuronosyltransferase (UGT) enzyme UGT1A6 inhibitor.

In some embodiments, the patient is also being administered a CYP2C8 inhibitor. In some embodiments, the patient is also being administered a CYP2C9 inhibitor. In some embodiments, the patient is also being administered a UDP-glucuronosyltransferase (UGT) enzyme UGT1A1 inhibitor. In some embodiments, the patient is also being administered a UDP-glucuronosyltransferase (UGT) enzyme UGT1A6 inhibitor.

In some embodiments, the CYP2C8 inhibitor is gemfibrozil, ritonavir, clopidogrel, lopinavir, deferasirox, lapatinib, trimethoprim, thiazolidinediones, montelukast, quercetin, candesartan cilexetil (cyclohexylcarbonate ester prodrug of candesartan), zafirlukast, clotrimazole, felodipine, mometasone firoate, salmetcrol, raloxifene, fenofibrate, ritonavir, levothyroxine, tamoxifen, loratadine, oxybutynin, medroxyprogesterone, simvastatin, ketoconazole, ethinyl estradiol, spironolactone, lovastatin, nifedipine, or irbesartan. In some embodiments, the CYP2C8 inhibitor is gemfibrozil.

In some embodiments, the CYP2C9 inhibitor is amiodarone, disulfram, doxifluridine, efavirenz, fluconazole, imatinib, lefluonomide, metronidazole, miconazole, phenytoin, sulfamethoxazole, sulfapenazone, sildenafil, zafirlukast, valdecoxib, diclofenac, voriconazole, tamoxifen, losartan, warfarin, etodolac, mefenamic acid, meloxicam, suprofen, irbesartan, ibuprofen, fluvastatin, sertraline, fluvoxamine, pantoprazole, rosiglitazone, lansoprazole, ritonavir, nicardipine, aprepitant, delavirdine, desloratadine, glyburide, ketoconazole, gemfibrozil, acenocoumarol, avasimibe, rosuvastatin, lovastatin, simvastatin, atorvastatin, cerivastatin, quinine, clozapine, or diazepam. In some embodiments, the CYP2C9 inhibitor is fluconazole. In some embodiments, the CYP2C9 inhibitor is gemfibrozil.

In some embodiments, the CYP2C9 inhibitor is ticlopidine, omeprazole, paroxetine, valsartan, bortezomib, valproic acid, nevirapine, azelastine, lornoxicam, fenofibrate, isoniazid, phenylbutazone, probenecid, sulfaphenazole, teniposide, etravirine, sulfadiazine, sulfinpyrazone, sulfisoxazole, trimethoprim, leflunomide, nilotinib, pyrimethamine, sorafenib, capecitabine, fluorouracil, sitaxentan, tranylcypromine, aminophenazone, clopidogrel, verapamil, etoricoxib, propofol, ketoprofen, seratrodast, sulfamoxole, amlodipine, amodiaquine, anastrozole, atovaquone, chloramphenicol, cyclosporine, cinetidine, clotrimazole cocaine, colchicine, cholecalciferol cyclizine, dexfenfluramine, dextropropoxyphene, dicoumarol, diltiazem, disulfiram, epinephrine, eprosartan, ethanol, felodipine, flecainide, histamine, indinavir, lopinavir, loratadine, medroxyprogesterone acetate, methazolamide, moclobemide, modafinil, nelfinavir, nifedipine, nilutamide, nilvadipine, olanzapine, phentermine, pioglitazone, pranlukast, pravastatin, promethazine, propafenone, quinidine, rutin, saquinavir, selegiline, sulfadimethoxine, sulfamethizole, sulfanilamide, sulfapyridine, tegaserod, methimazole, thioridazine, tioconazole, tolcapone, triazolam, troglitazone, bicalutamide, rabeprazole, armodafinil, diethylstilbestrol, agomelatine, noscapine, clevidipine, cisplatin, human serum albumin, sulconazole, vismodegib, regorafenib, gefitinib, parecoxib, lumacaftor, abiraterone, ticagrelor, ceritinib, floxuridine, crisaborole midostaurin, belinostat, lifitegrast, rhein, diacerein, doconexent, topiroxostat, zucapsaicin, stiripentol, lobeglitazone, dosulepin, benzbronarone, manidipine, enasidenib, candesartan, rucaparib, isavuconazole, cimicifuga racemose, nabilone, acetyl sulfisoxazole, or curcumin.

In some embodiments, the UDP-glucuronosyltransferase (UGT) enzyme UGT1A1 inhibitor is adenine, propofol, indomethacin, nilotinib, pazopanib, regorafenib, flunitrazepam, erlotinib, sorafenib, enasidenib, pibrentasvir, glecaprevir, rucaparib, ertugliflozin, or fostamatinib.

In some embodiments, the UDP-glucuronosyltransferase (UGT) enzyme UGT1A6 inhibitor is troglitazone.

In some embodiments, the UGT substrate is diclofenac.

Also provided is a method of administering a sphingosine 1-phosphate subtype 1 (S1P₁) receptor modulator chosen from (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to a patient in need thereof wherein the patient is also being administered a substrate of a membrane transporter wherein the membrane transporter is selected from P-glycoprotein (Pgp), BCRP (breast cancer resistance protein), and OATP1B1, comprising: administering to the patient a therapeutically effective amount of the S1P₁ modulator.

Also provided is a method of administering a sphingosine 1-phosphate subtype 1 (S1P₁) receptor modulator chosen from R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt and/or isotopic variant thereof, to a patient in need thereof, comprising: administering to the patient a therapeutically effective amount of the S1P₁ receptor modulator, subsequently determining that the patient is to begin treatment with a substrate of a membrane transporter wherein the membrane transporter is selected from P-glycoprotein (Pgp), BCRP (breast cancer resistance protein), and OATP1B1, and continuing administration of the therapeutically effective amount of the S1P₁ receptor modulator to the patient.

In some embodiments, the method further comprises monitoring the patient for signs and symptoms of toxicity and clinical response associated with the substrate of the membrane transporter.

In some embodiments, the method further comprises reducing the amount of the substrate of the membrane transporter administered to the patient based on the patient's ability to tolerate one or more exposure-related adverse reactions related to the substrate of the membrane transporter.

In some embodiments, the method further comprises informing the patient or a medical care worker that co-administration of the S1P₁ receptor modulator and the substrate of a membrane transporter may result in increased exposure of the substrate of the membrane transporter.

In some embodiments, the method further comprises informing the patient or a medical care worker that co-administration of the S1P₁ receptor modulator and the substrate of the membrane transporter may result in increased risk of one or more exposure-related adverse reactions associated with the substrate of the membrane transporter.

In some embodiments, monitoring for signs and symptoms of toxicity and clinical response comprises monitoring the serum concentration of the substrate of the membrane transporter.

In some embodiments, monitoring for signs and symptoms of toxicity and clinical response comprises determining whether the patient experiences one or more exposure-related adverse reaction associated with serum concentration of the substrate of the membrane transporter.

In some embodiments, monitoring for signs and symptoms of toxicity and clinical response comprises monitoring efficacy of the substrate of the membrane transporter.

In some embodiments, the membrane transporter is P-glycoprotein (Pgp). In some embodiments, the membrane transporter is BCRP (breast cancer resistance protein). In some embodiments, the membrane transporter is OATP1B1.

In some embodiments, the membrane transporter is P-glycoprotein. In some embodiments, the substrate of the membrane transporter is chosen from digoxin, loperamide, berberine, irinotecan, doxorubicin, vinblastine, paclitaxel, and fexofenadine.

In some embodiments, the membrane transporter is BCRP. In some embodiments, the substrate of the membrane transporter is chosen from mitoxantrone, methotrexate, topotecan, imatinib, irinotecan, statins, sulphate conjugates, and porphyrins.

In some embodiments, the membrane transporter is OATP1B1. In some embodiments, the substrate of the membrane transporter is chosen from bromosulphophthalein, oestrone-3-sulphate, oestradiol-170-glucuronide, statins, repaglinide, valsartan, olmesartan, bilirubin glucuronide, bilirubin, and bile acids. In some embodiments, the substrate of the membrane transporter is rifampin.

In some embodiments, the membrane transporter is OATP1B3. In some embodiments, the substrate of the membrane transporter is rifampin.

In some embodiments, the dosage form is administered under fasted conditions. In some embodiments, the dosage form is administered under fed conditions.

In some embodiments, the method is non-gender specific.

In some embodiments, the individual is also being administered one or more agents independently chosen from oral corticosteroids and aminosalicylates.

In some embodiments, the individual is not being administered one or more agents independently chosen from natalizumab, efalizumab, and rituximab. In some embodiments, the administration of Compound 1 is not initiated if the individual is being administered one or more agents independently chosen from natalizumab, efalizumab, and rituximab. In some embodiments, the administration of Compound 1 is discontinued if the individual is being administered one or more agents independently chosen from natalizumab, efalizumab, and rituximab. In some embodiments, the dose of Compound 1 is reduced if the individual is being administered one or more agents independently chosen from natalizumab, efalizumab, and rituximab. In some embodiments, the individual has not been administered a biologic agent. In some embodiments, the individual has not been administered two or more biologic agents. In some embodiments, the individual has not been administered three or more biologic agents. In some embodiments, the individual is not being administered a biologic agent. In some embodiments, the individual is not being administered two or more biologic agents. In some embodiments, the individual is not being administered three or more biologic agents.

In some embodiments, the standard dose is administered without titration; and the individual does not experience a severe related adverse event.

In some embodiments, the therapeutically effective amount is equivalent to about 0.5 to about 5.0 mg of Compound 1 if the individual does not have an active infection. In some embodiments, Compound 1 is not administered to the individual 1 if the individual has an active infection. In some embodiments, the active infection is a serious active infection. In some embodiments, the method further comprises monitoring the individual for an active infection. In some embodiments, the method further comprises discontinuing administration if the individual develops an active infection.

In some embodiments, the method further comprises monitoring for adverse events during the administration of Compound 1, or a pharmaceutically acceptable salt, hydrate, or solvate thereof, and optionally, interrupting or terminating the administration of Compound 1, or a pharmaceutically acceptable salt, hydrate, or solvate thereof.

In some embodiments, the treatment further comprises monitoring heart rate during the administration, monitoring pulmonary function during the administration, or monitoring liver function during the administration.

In some embodiments, the treatment further comprises monitoring heart rate during the administration.

In some embodiments, the treatment further comprises monitoring pulmonary function during the administration.

In some embodiments, the treatment further comprises monitoring liver function during the administration.

In some embodiments, the method reduces the incidence and severity of adverse events resulting from the treatment of the sphingosine 1-phosphate subtype 1 (S1P₁) receptor-associated disorder.

In some embodiments, the adverse event is a serious adverse event.

In some embodiments, the serious adverse event is selected from leukopenia, constipation, diarrhea, nausea, abdominal pain, neutropenia, vomiting, back pain, and menstrual disorder.

In some embodiments, the method results in no serious adverse events.

In some embodiments, the standard dose is administered without substantially inducing an acute heart rate reduction or heart block in the individual.

In some embodiments, Compound 1 is administered without causing a reduction of more than 6 bpm in heart rate.

In some embodiments, Compound 1 is administered without a first-dose effect on heart rate as seen with other S1P receptor modulators. In some embodiments, Compound 1 is administered without a first-dose effect on AV conduction as seen with other S1P receptor modulators.

In some embodiments, the individual was previously administered at least one agent selected from: a TNF antagonist, an integrin antagonist, and an immunosuppressive agent.

In some embodiments, the individual had an inadequate response with, lost response to, or was intolerant to the at least one agent.

In some embodiments, the individual had demonstrated, over the previous 3-month period, an inadequate response to, loss of response to, or intolerance of at least one agent selected from oral 5-aminosalicylates, corticosteroids, immunosuppressives, TNFα antagonists, and integrin antagonists. In some embodiments, the individual had demonstrated, over the previous 6-month period, an inadequate response to, loss of response to, or intolerance of at least one agent selected from oral 5-aminosalicylates, corticosteroids, immunosuppressives, TNFα antagonists, and integrin antagonists. In some embodiments, the individual had demonstrated, over the previous 9-month period, an inadequate response to, loss of response to, or intolerance of at least one agent selected from oral 5-aminosalicylates, corticosteroids, immunosuppressives, TNFα antagonists, and integrin antagonists. In some embodiments, the individual had demonstrated, over the previous 1-year period, an inadequate response to, loss of response to, or intolerance of at least one agent selected from oral 5-aminosalicylates, corticosteroids, immunosuppressives, TNFα antagonists, and integrin antagonists. In some embodiments, the individual had demonstrated, over the previous 2-year period, an inadequate response to, loss of response to, or intolerance of at least one agent selected from oral 5-aminosalicylates, corticosteroids, immunosuppressives, TNFα antagonists, and integrin antagonists. In some embodiments, the individual had demonstrated, over the previous 3-year period, an inadequate response to, loss of response to, or intolerance of at least one agent selected from oral 5-aminosalicylates, corticosteroids, immunosuppressives, TNFα antagonists, and integrin antagonists. In some embodiments, the individual had demonstrated, over the previous 4-year period, an inadequate response to, loss of response to, or intolerance of at least one agent selected from oral 5-aminosalicylates, corticosteroids, immunosuppressives, TNFα antagonists, and integrin antagonists. In some embodiments, the individual had demonstrated, over the previous 5-year period, an inadequate response to, loss of response to, or intolerance of at least one agent selected from oral 5-aminosalicylates, corticosteroids, immunosuppressives, TNFα antagonists, and integrin antagonists.

In some embodiments, the standard dose is administered without titration.

In some embodiments, the individual has fasted prior to being administered the standard dose

In some embodiments, treating comprises inducing and/or maintaining clinical response; improving endoscopic appearance of the mucosa; and/or inducing and/or maintaining clinical remission.

In some embodiments, treating comprises inducing and/or maintaining histologic improvement.

In some embodiments, treating comprises inducing and/or maintaining histologic remission.

In some embodiments, treating comprises inducing and/or maintaining mucosal healing.

In some embodiments, prior to the administering the individual has a 3-component Mayo Clinic Score of at least 6.

In some embodiments, the method results in an improvement of the individual's 3-component Mayo Clinic Score. In some embodiments, the method results in an improvement of the individual's 2-component Mayo Clinic Score. In some embodiments, the method results in an improvement of the individual's Total Mayo Clinic Score.

In some embodiments of the method of treatment of inflammatory bowel disease, e.g., ulcerative colitis, such as moderately to severely active ulcerative colitis, the treatment results in endoscopic improvement, e.g., improving endoscopic appearance of the mucosa.

In some embodiments of the method of treatment of inflammatory bowel disease, e.g., ulcerative colitis, such as moderately to severely active ulcerative colitis, the treatment results in inducing clinical remission. In some embodiments of the method of treatment of inflammatory bowel disease, e.g., ulcerative colitis, such as moderately to severely active ulcerative colitis, the treatment results in maintaining clinical remission. In some embodiments of the method of treatment of inflammatory bowel disease, e.g., ulcerative colitis, such as moderately to severely active ulcerative colitis, the treatment results in inducing and maintaining clinical remission.

In some embodiments of the method of treatment of inflammatory bowel disease, e.g., ulcerative colitis, such as moderately to severely active ulcerative colitis, the treatment results in inducing clinical response. In some embodiments of the method of treatment of inflammatory bowel disease, e.g., ulcerative colitis, such as moderately to severely active ulcerative colitis, the treatment results in maintaining clinical response. In some embodiments of the method of treatment of inflammatory bowel disease, e.g., ulcerative colitis, such as moderately to severely active ulcerative colitis, the treatment results in inducing and maintaining clinical response.

In some embodiments, the treatment reduces a lymphocyte count in the individual by at least 40%. In some embodiments, the treatment reduces a lymphocyte count in the individual by at least 45%, 50%, 55%, 60%, or 65%.

In some embodiments of the method of treatment of inflammatory bowel disease, e.g., ulcerative colitis, such as moderately to severely active ulcerative colitis, the treatment results in corticosteroid-free remission.

In some embodiments of the method of treatment of inflammatory bowel disease, e.g., ulcerative colitis, such as moderately to severely active ulcerative colitis, the treatment results in endoscopic remission.

In some embodiments of the method of treatment of inflammatory bowel disease, e.g., ulcerative colitis, such as moderately to severely active ulcerative colitis, the treatment results in an improvement in rectal bleeding.

In some embodiments of the method of treatment of inflammatory bowel disease, e.g., ulcerative colitis, such as moderately to severely active ulcerative colitis, the treatment results in histologic healing.

In some embodiments of the method of treatment of inflammatory bowel disease, e.g., ulcerative colitis, such as moderately to severely active ulcerative colitis, the treatment results in an improvement in stool frequency.

In some embodiments of the method of treatment of inflammatory bowel disease, e.g., ulcerative colitis, such as moderately to severely active ulcerative colitis, the treatment further comprises monitoring the level of level of fecal calprotectin.

In some embodiments of the method of treatment of inflammatory bowel disease, e.g., ulcerative colitis, such as moderately to severely active ulcerative colitis, the treatment further comprises monitoring the level of level of c-reactive protein (CRP).

In some embodiments, treating is reducing a sign and/or symptom of ulcerative colitis. In some embodiments, treating is reducing a sign of ulcerative colitis. In some embodiments, treating is reducing a symptom of ulcerative colitis. In some embodiments, treating is reducing a sign and/or symptom of Crohn's disease. In some embodiments, treating is reducing a sign of Crohn's disease. In some embodiments, treating is reducing a symptom of Crohn's disease.

In some embodiments, treating is inducing and/or maintaining clinical remission. In some embodiments, treating is inducing and maintaining clinical remission. In some embodiments, treating is inducing and/or maintaining clinical remission and/or clinical response. In some embodiments, treating is inducing and maintaining clinical remission and clinical response. In some embodiments, treating is inducing clinical remission and/or clinical response. In some embodiments, treating is maintaining clinical remission and/or clinical response. In some embodiments, treating is inducing clinical remission and clinical response. In some embodiments, treating is maintaining clinical remission and clinical response. In some embodiments, treating is inducing and/or maintaining clinical remission and/or mucosal healing. In some embodiments, treating is inducing and maintaining clinical remission and mucosal healing. In some embodiments, treating is inducing and maintaining mucosal healing. In some embodiments, treating is inducing and maintaining clinical remission. In some embodiments, treating is inducing clinical remission. In some embodiments, treating is inducing mucosal healing. In some embodiments, treating is maintaining clinical remission. In some embodiments, treating is maintaining mucosal healing. In some embodiments, treating is achieving and/or sustaining clinical remission in induction responders. In some embodiments, treating is achieving and sustaining clinical remission in induction responders. In some embodiments, treating is achieving clinical remission in induction responders. In some embodiments, treating is sustaining clinical remission in induction responders. In some embodiments, treating is inducing and/or maintaining clinical response. In some embodiments, treating is inducing and maintaining clinical response. In some embodiments, treating is inducing clinical response. In some embodiments, treating is maintaining clinical response. In some embodiments, treating is inducing endoscopic improvement. In some embodiments, treating is maintaining endoscopic improvement. In some embodiments, treating is achieved endoscopic improvement. In some embodiments, treating is improving endoscopic remission. In some embodiments, treating is maintaining endoscopic remission. In some embodiments, treating is inducing histologic healing. In some embodiments, treating is maintaining histologic healing. In some embodiments, treating is improving stool frequency. In some embodiments, treating is maintaining improvement in stool frequency. In some embodiments, treating is improving endoscopic appearance of the mucosa. In some embodiments, treating is maintaining endoscopic improvement of the mucosa. In some embodiments, treating is improving endoscopic appearance of the mucosa during induction. In some embodiments, treating eliminates the need for corticosteroid use. In some embodiments, treating allows for reduced corticosteroid use. In some embodiments, treating allows for the use of a lower dose of a corticosteroid. In some embodiments, treating is achieving corticosteroid-free remission. In some embodiments, treating is sustaining corticosteroid-free remission. In some embodiments, treating is improving rectal bleeding. In some embodiments, treating is maintaining improvement in rectal bleeding. In some embodiments, treating is improving endoscopic subscore. In some embodiments, treating is maintaining improvement in endoscopic subscore.

In some embodiments, ulcerative colitis has been diagnosed using a 2-component Mayo Clinic Score. For example, in some embodiments, ulcerative colitis has been diagnosed using a score ranging from 0 to 9 for rectal bleeding and endoscopic findings. In some embodiments, ulcerative colitis has been diagnosed using a 3-component Mayo Clinic Score. For example, in some embodiments, ulcerative colitis has been diagnosed using a score ranging from 0 to 9 for stool frequency, rectal bleeding, and endoscopic findings. In some embodiments, ulcerative colitis has been diagnosed using a Total Mayo Score. For example, in some embodiments, ulcerative colitis has been diagnosed using a score ranging from 0 to 12 for stool frequency, rectal bleeding, endoscopic findings, and Physicians Global Assessment.

In some embodiments, improvement in ulcerative colitis is measured using a 2-component Mayo Clinic Score. In some embodiments, improvement in ulcerative colitis is measured using a 3-component Mayo Clinic Score. In some embodiments, improvement in ulcerative colitis is measured using a Total Mayo Score. In some embodiments, improvement in ulcerative colitis is measured by clinical remission. In some embodiments, improvement in ulcerative colitis is measured by lymphocyte reduction. In some embodiments, improvement in ulcerative colitis is measured by endoscopic improvement. In some embodiments, improvement in ulcerative colitis is measured by 6-point Mayo Score. For example, in some embodiments, improvement in ulcerative colitis is measured by stool frequency and rectal bleeding. In some embodiments, improvement in ulcerative colitis is statistically significant.

In some embodiments, Compound 1 is not recommended in an individual with active, severe infection. In some embodiments, Compound 1 is not recommended in an individual with an active infection. In some embodiments, Compound 1 is not recommended in an individual with a severe infection. In some embodiments, Compound 1 is not recommended in an individual with an active, severe infection until the infection is controlled. In some embodiments, Compound 1 is not recommended in an individual with an active infection until the infection is controlled. In some embodiments, Compound 1 is not recommended in an individual with a severe infection until the infection is controlled. In some embodiments, administration of Compound 1 is not started during an active infection. In some embodiments, an individual is monitored for infection. In some embodiments, administration of Compound 1 is stopped if an individual develops an infection. In some embodiments, administration of Compound 1 is stopped if infection becomes serious. In some embodiments, administration of Compound 1 is discontinued if an individual develops an infection. In some embodiments, Compound 1 is not administered to an individual with an infection. In some embodiments, Compound 1 is not administered during an active infection. In some embodiments, administration of Compound 1 is not started during active infection; an individual is monitored if an infection develops during administration; and administration is stopped if the infection becomes serious. In some embodiments, an infection is mild. In some embodiments, an infection is moderate. In some embodiments, an infection is severe. In some embodiments, an infection is serious. In some embodiments, an infection is a serious adverse event. In some embodiments, an infection is a respiratory infection.

In some embodiments, Compound 1 is administered without causing a severe adverse event. In some embodiments, Compound 1 is administered without causing a severe adverse event related to heart rate. In some embodiments, Compound 1 is administered without causing a severe adverse event related to heart rate change. In some embodiments, Compound 1 is administered without causing a severe adverse event related to elevated heart rate. In some embodiments, Compound 1 is administered without causing a severe adverse event related to bradycardia. In some embodiments, Compound 1 is administered without causing a severe adverse event related to AV block. In some embodiments, Compound 1 is administered without causing a severe adverse event related to AV conduction. In some embodiments, Compound 1 is administered without causing bradycardia. In some embodiments, Compound 1 is administered without causing AV block. In some embodiments, Compound 1 is administered without causing more than mild decrease in heart rate on first day of treatment (for example, >10 bpm). In some embodiments, Compound 1 is administered without a first-dose effect seen with other S1P receptor modulators. In some embodiments, Compound 1 is administered without a first-dose cardiovascular effect seen with other S1P receptor modulators. In some embodiments, Compound 1 is administered without symptomatic changes in heart rate. In some embodiments, Compound 1 is administered without symptomatic changes in heart rhythm. In some embodiments, Compound 1 is administered without requiring titration to avoid first-dose effect seen with other S1P receptor modulators.

In some embodiments, Compound 1 is administered without increasing a liver function test (LFT). In some embodiments, Compound 1 is administered without causing an elevated LFT. In some embodiments, Compound 1 is administered without increasing ALT. In some embodiments, Compound 1 is administered without increasing AST. In some embodiments, Compound 1 is administered without increasing ALT>3×ULN. In some embodiments, Compound 1 is administered without increasing ALT>2.5×ULN. In some embodiments, Compound 1 is administered without increasing ALT>2×ULN. In some embodiments, Compound 1 is administered without increasing ALT>1.5×ULN. In some embodiments, Compound 1 is administered without increasing AST>3×ULN. In some embodiments, Compound 1 is administered without increasing AST>2.5×ULN. In some embodiments, Compound 1 is administered without increasing AST>2×ULN. In some embodiments, Compound 1 is administered without increasing AST>1.5×ULN. In some embodiments, Compound 1 is administered without increasing bilirubin. In some embodiments, Compound 1 is administered without increasing bilirubin >3×ULN. In some embodiments, Compound 1 is administered without increasing bilirubin >2.5×ULN. In some embodiments, Compound 1 is administered without increasing bilirubin >2×ULN. In some embodiments, Compound 1 is administered without increasing bilirubin >1.5×ULN. In some embodiments, Compound 1 is administered without increasing gamma-glutamyl transferase (GGT). In some embodiments, Compound 1 is administered without increasing GGT>3×ULN. In some embodiments, Compound 1 is administered without increasing GGT>2.5×ULN. In some embodiments, Compound 1 is administered without increasing GGT>2×ULN. In some embodiments, Compound 1 is administered without increasing GGT>1.5×ULN.

In some embodiments, Compound 1 is administered without causing an abnormality in a pulmonary function test. In some embodiments, Compound 1 is administered without causing macular edema.

In some embodiments, the individual has had an inadequate response with, lost response to, been intolerant to, or demonstrated dependence on another agent for the treatment of an inflammatory bowel disease. In some embodiments, the individual has had an inadequate response with the other agent for the treatment of an inflammatory bowel disease. In some embodiments, the individual has lost response to another agent for the treatment of an inflammatory bowel disease. In some embodiments, the individual was intolerant to another agent for the treatment of an inflammatory bowel disease. In some embodiments, the individual requires continuous steroid therapy. In some embodiments, the other agent is at least one agent selected from: a tumor necrosis tumor necrosis factor (TNF) antagonist, a corticosteroid, an integrin antagonist, and immunosuppressive agent, and an aminosalicylate.

In some embodiments, the individual has had an inadequate response with, lost response to, or been intolerant to a conventional therapy. In some embodiments, the individual has had an inadequate response to conventional therapy. In some embodiments, the individual has lost response to conventional therapy. In some embodiments, the individual has been intolerant to conventional therapy. In some embodiments, the conventional therapy is selected from: at least one agent selected from: a tumor necrosis tumor necrosis factor (TNF) antagonist, a corticosteroid, an integrin antagonist, and immunosuppressive agent, and an aminosalicylate.

In some embodiments, the individual was previously administered a corticosteroid and/or an aminosalicylate. In some embodiments, the individual was previously administered a tumor necrosis tumor necrosis factor (TNF) antagonist, an integrin antagonist, and/or an immunosuppressive agent.

In some embodiments, the corticosteroid is an oral corticosteroid. In some embodiments, the TNF antagonist is a TNF-α blocker. In some embodiments, the aminosalicylate is a 5-aminosalicylate. In some embodiments, the integrin antagonist is referred to as an integrin receptor antagonist. In some embodiments, the TNF antagonist is referred to as a TNF blocker. In some embodiments, the immunosuppressive agent is referred to as an immunomodulator. In some embodiments, the prior conventional therapy is referred to as prior treatment.

In some embodiments, the individual is not administered a therapeutic dose of a thiopurine. In some embodiments, the individual is not administered a therapeutic dose of azathioprine. In some embodiments, the individual is not administered a therapeutic dose of 6-mercaptopurine. In some embodiments, the individual is not administered a therapeutic dose of thioguanine (also referred to as tioguanine or 6-thioguanine).

In some embodiments, the inhibitor is a moderate inhibitor. In some embodiments, the inhibitor is a strong inhibitor. In some embodiments, the inducer is a moderate inducer. In some embodiments, the inducer is a strong inducer.

In some embodiments, caution is used when Compound 1 is co-administered with a CYP substrate. In some embodiments, caution is used when Compound 1 is co-administered with a UGT substrate. In some embodiments, caution is used when Compound 1 is co-administered with an OAT substrate. In some embodiments, caution is used when Compound 1 is co-administered with strong inhibitors. In some embodiments, the strong inhibitor is a strong CYP inhibitor. In some embodiments, the strong inhibitor is a CYP2C8. In some embodiments, caution is used when Compound 1 is co-administered with moderate inhibitors. In some embodiments, the moderate inhibitor is a moderate CYP inhibitor. In some embodiments, the moderate inhibitor is a CYP2C9 inhibitor. In some embodiments, caution is used when Compound 1 is co-administered with strong inducers. In some embodiments, the strong inhibitor is a strong CYP inducer. In some embodiments, caution is used when Compound 1 is co-administered with moderate inducers. In some embodiments, the moderate inducer is a moderate CYP inhibitor. In some embodiments, the moderate inducer is a CYP2C8 inducer. In some embodiments, the moderate inducer is a CYP2C9 inducer.

In some embodiments, the dose of Compound 1 is limited when used with a CYP substrate. In some embodiments, the dose of Compound 1 is limited when used a UGT substrate. In some embodiments, the dose of Compound 1 is limited when used with an OAT substrate. In some embodiments, the dose of Compound 1 is limited when used with strong inhibitors. In some embodiments, the dose of Compound 1 is limited to, or is limited to about, does not exceed, or does not exceed about, 0.25, 0.3, 0.4, 0.5, 0.6, 0.7. 0.75, 0.8, 0.9, 1.0., 1.1., 1.2, 1.3, 1.4, 1.5, 1.6, 1.7, 1.75, 1.8, 1.9, or 2.0 mg. In some embodiments, Compound 1 is administered at the lowest dose when used with a strong inhibitor. In some embodiments, the dose of Compound 1 is limited when used with moderate inhibitors. In some embodiments, Compound 1 is administered at the lowest dose when used with a moderate inhibitor. In some embodiments, the lowest dose is the lowest efficacious dose. In some embodiments, the lowest dose is the lowest marketed dose. In some embodiments, the lowest dose is the lowest marketed dose in the United States. In some embodiments, the lowest dose of Compound 1 is, or is about, 0.25, 0.3, 0.4, 0.5, 0.6, 0.7. 0.75, 0.8, 0.9, 1.0., 1.1., 1.2, 1.3, 1.4, 1.5, 1.6, 1.7, 1.75, 1.8, 1.9, or 2.0 mg.

In some embodiments, the dose of a co-administered compound is limited when used with Compound 1. In some embodiments, the co-administered compound is a CYP substrate. In some embodiments, the co-administered compound is a UGT substrate. In some embodiments, the dose of a co-administered compound is an OAT substrate. In some embodiments, the co-administered compound is a strong inducer. In some embodiments, the co-administered compound is a strong inhibitor. In some embodiments, the co-administered compound is a moderate inducer. In some embodiments, the co-administered compound is a moderate inhibitor. In some embodiments, the co-administered compound is administered at the lowest dose. In some embodiments, the lowest dose is the lowest efficacious dose. In some embodiments, the lowest dose is the lowest marketed dose. In some embodiments, the lowest dose is the lowest marketed dose in the United States.

In some embodiments, Compound 1 is not used with a CYP substrate. In some embodiments, Compound 1 is not used with a UGT substrate. In some embodiments, Compound 1 is not used with an OAT substrate. In some embodiments, Compound 1 is not used with strong inducers. In some embodiments, Compound 1 is not used with moderate inducers. In some embodiments, Compound 1 is not used with strong inhibitors. In some embodiments, Compound 1 is not used with moderate inhibitors.

In some embodiments, concomitant use of Compound 1 is not recommended with a CYP substrate. In some embodiments, concomitant use of Compound 1 is not recommended with a UGT substrate. In some embodiments, concomitant use of Compound 1 is not recommended with an OAT substrate. In some embodiments, concomitant use of Compound 1 is not recommended with strong inducers. In some embodiments, concomitant use of Compound 1 is not recommended with moderate inducers. In some embodiments, concomitant use of Compound 1 is not recommended with strong inhibitors. In some embodiments, concomitant use of Compound 1 is not recommended with moderate inhibitors. In some embodiments, concomitant use of Compound 1 is not recommended with a CYP2C8 inhibitor. In some embodiments, concomitant use of Compound 1 is not recommended with a CYP2C8 inducer. In some embodiments, concomitant use of Compound 1 is not recommended with a CYP2C9 inhibitor. In some embodiments, concomitant use of Compound 1 is not recommended with a CYP2C8 inducer.

Some embodiments provide a method of safely treating an individual with (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof.

Some embodiments provide a method of safely administering (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof.

Some embodiments provide a method of administering a daily dose of Compound 1 when an individual is concomitantly receiving a CYP substrate, OAT substrate, or UGT substrate. In some embodiments, the daily dose of Compound 1 is nor more than, or no more than about, 0.25, 0.3, 0.4, 0.5, 0.6, 0.7. 0.75, 0.8, 0.9, 1.0., 1.1., 1.2, 1.3, 1.4, 1.5, 1.6, 1.7, 1.75, 1.8, 1.9, 2.0, 2.1, 2.2, 2.25, 2.3, 2.4, 2.5, 2.6, 2.7, 2.75, 2.8, 2.9, or 3.0 mg. In some embodiments, the daily dose of Compound 1 is less than, or less than about, 0.25, 0.3, 0.4, 0.5, 0.6, 0.7. 0.75, 0.8, 0.9, 1.0., 1.1., 1.2, 1.3, 1.4, 1.5, 1.6, 1.7, 1.75, 1.8, 1.9, 2.0, 2.1, 2.2, 2.25, 2.3, 2.4, 2.5, 2.6, 2.7, 2.75, 2.8, 2.9, or 3.0 mg.

In some embodiments, the CYP substrate is a CYP2C8 substrate.

In some embodiments, the CYP substrate is a CYP2C9 substrate.

In some embodiments, the UGT substrate is a UGT1A1 substrate.

In some embodiments, the UGT substrate is a UGT1A4 substrate.

In some embodiments, the UGT substrate is a UGT1A6 substrate.

In some embodiments, the UGT substrate is a UGT1A7 substrate.

In some embodiments, the OAT substrate is an OATP1B1 substrate.

In some embodiments, the OAT substrate is an OATP1B3 substrate.

In some embodiments, the OAT substrate is an OAT1 substrate.

In some embodiments, the OAT substrate is an OAT3 substrate.

In some embodiments, the CYP2C8 inhibitor, CYP2C9 inhibitor, CYP2C8 inducer, or CYP2C9 inducer is fluconazole.

In some embodiments, the CYP2C8 inhibitor, CYP2C9 inhibitor, CYP2C8 inducer, or CYP2C9 inducer is gemfibrozil.

In some embodiments, the CYP2C8 inhibitor, CYP2C9 inhibitor, CYP2C8 inducer, or CYP2C9 inducer is rifampin.

In some embodiments, the substrate of OATP1B1 is rifampin.

In some embodiments, the substrate of OATP1B3 is rifampin.

In some embodiments, the co-administered compound is a CYP2C8 inhibitor.

In some embodiments, the co-administered compound is a CYP2C8 inducer.

In some embodiments, the co-administered compound is a CYP2C9 inhibitor.

In some embodiments, the co-administered compound is a CYP2C9 inducer.

In some embodiments, the co-administered compound is fluconazole.

In some embodiments, the co-administered compound is gemfibrozil.

In some embodiments, the co-administered compound is rifampin.

In some embodiments, less than the amount that would be administered to a patient who is not also being administered a CYP substrate, OAT substrate, UGT substrate, CYP2C8 inhibitor, CYP2C9 inhibitor, CYP2C8 inducer, CYP2C9 inducer, UGT1A1 inhibitor, or UGT1A6 inhibitor is about, at least, or at least about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 778, 79, 80, 81, 82, 83, 84, 85, 86, 87, 88, 89, or 90% less than the amount that would be administered to a patient who is not also being administered a CYP substrate, OAT substrate, UGT substrate, CYP2C8 inhibitor, CYP2C9 inhibitor, CYP2C8 inducer, CYP2C9 inducer, UGT1A1 inhibitor, or UGT1A6 inhibitor.

In some embodiments, less than the amount that would be administered to a patient who is not also being administered a CYP substrate, OAT substrate, UGT substrate, CYP2C8 inhibitor, CYP2C9 inhibitor, CYP2C8 inducer, CYP2C9 inducer, UGT1A1 inhibitor, or UGT1A6 inhibitor is about, at least, or at least about, 0.1, 0.2, 0.25, 0.3, 0.4, 0.5, 0.6, 0.7, 0.75, 0.8, 0.9, 1.0, 1.1, 1.2, 1.25, 1.3, 1.4, 1.5, 1.6, 1.7, 1.75, 1.8, 1.9, or 2.0 mg less than the amount that would be administered to a patient who is not also being administered a CYP substrate, OAT substrate, UGT substrate, CYP2C8 inhibitor, CYP2C9 inhibitor, CYP2C8 inducer, CYP2C9 inducer, UGT1A1 inhibitor, or UGT1A6 inhibitor.

Some embodiments provide a method of safely administering a sphingosine 1-phosphate subtype 1 (S1P₁) receptor modulator chosen from (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof, to a patient in need thereof wherein the patient is also being administered a cytochrome P450 (CYP) inhibitor, CYP inducer, organic anion transporter (OAT) substrate, UDP-glucuronosyltransferase (UGT) enzyme inhibitor, or UDP-glucuronosyltransferase (UGT) enzyme inhibitor, comprising administering to the patient a daily dose of less than 2 mg of the S1P₁ modulator.

In some embodiments, the daily dose of the S1P₁ modulator is selected from: 1.0, 1.25, 1.5, and 1.75 mg of the S1P₁ modulator.

S1P receptor agonists having agonist activity on the S1P₁ receptor have been shown to rapidly and reversibly induce lymphopenia (also referred to as peripheral lymphocyte lowering (PLL); Hale et al., Bioorg. Med. Chem. Lett., 14:3351-3355, 2004). This is attended by clinically useful immunosuppression by virtue of sequestering T- and B-cells in secondary lymphoid tissue (lymph nodes and Peyer's patches) and thus apart from sites of inflammation and organ grafts (Rosen et al., Immunol. Rev., 195:160-177, 2003; Schwab et al., Nature Immunol., 8:1295-1301, 2007). This lymphocyte sequestration, for example in lymph nodes, is thought to be a consequence of concurrent agonist-driven functional antagonism of the S1P₁ receptor on T-cells (whereby the ability of S1P to mobilize T-cell egress from lymph nodes is reduced) and persistent agonism of the S1P₁ receptor on lymph node endothelium (such that barrier function opposing transmigration of lymphocytes is increased) (Matloubian et al., Nature, 427:355-360, 2004; Baumruker et al., Expert Opin. Investig. Drugs, 16:283-289, 2007). It has been reported that agonism of the S1P₁ receptor alone is sufficient to achieve lymphocyte sequestration (Sanna et al., J. Biol. Chem., 279:13839-13848, 2004) and that this occurs without impairment of immune responses to systemic infection (Brinkmann et al., Transplantation, 72:764-769, 2001; Brinkmann et al., Transplant. Proc., 33:530-531, 2001).

That agonism of endothelial S1P₁ receptors has a broader role in promoting vascular integrity is supported by work implicating the S1P₁ receptor in capillary integrity in mouse skin and lung (Sanna et al., Nat. Chem. Biol., 2:434-441, 2006). Vascular integrity can be compromised by inflammatory processes, for example as may derive from sepsis, major trauma and surgery so as to lead to acute lung injury or respiratory distress syndrome (Johan Groeneveld, Vascul. Pharmacol., 39:247-256, 2003).

An exemplary S1P receptor agonist having agonist activity on the S1P₁ receptor is FTY720 (fingolimod), an immunosuppressive agent that has undergone clinical trials (Martini et al., Expert Opin. Investig. Drugs, 16:505-518, 2007) and was recently approved by the FDA for the treatment of individuals with relapsing forms of multiple sclerosis (MS) to reduce the frequency of clinical exacerbations and to delay the accumulation of physical disability. FTY720 acts as a prodrug which is phosphorylated in vivo; the phosphorylated derivative is an agonist for S1P₁, S1P₃, S1P₄ and S1P₅ receptors (but not the S1P₂ receptor) (Chiba, Pharmacology & Therapeutics, 108:308-319, 2005). FTY720 has been shown to rapidly and reversibly induce lymphopenia; Hale et al., Bioorg. Med. Chem. Lett., 14:3351-3355, 2004). This is attended by clinically useful immunosuppression by virtue of sequestering T- and B-cells in secondary lymphoid tissue (lymph nodes and Peyer's patches) and thus apart from sites of inflammation and organ grafts (Rosen et al., Immunol. Rev., 195:160-177, 2003; Schwab et al., Nature Immunol., 8:1295-1301, 2007).

In clinical trials, FTY720 elicited an adverse event (i.e., transient asymptomatic bradycardia) which may be due to its agonism of the S1P₃ receptor (Budde et al., J. Am. Soc. Nephrol., 13:1073-1083, 2002; Sanna et al., J. Biol. Chem., 279:13839-13848, 2004; Ogawa et al., BBRC, 361:621-628, 2007).

FTY720 has been reported to have therapeutic efficacy in at least: a rat model for autoimmune myocarditis and a mouse model for acute viral myocarditis (Kiyabayashi et al., J Cardiovasc. Pharmacol., 35:410-416, 2000; Miyamoto et al., J Am. Coll. Cardiol., 37:1713-1718, 2001); mouse models for inflammatory bowel disease including colitis (Mizushima et al., Inflamm. Bowel Dis., 10:182-192, 2004; Deguchi et al., Oncology Reports, 16:699-703, 2006; Fujii et al., Am. J Physiol. Gastrointest. Liver Physiol., 291:G267-G274, 2006; Daniel et al., J Immunol., 178:2458-2468, 2007); a rat model for progressive mesangioproliferative glomerulonephritis (Martini et al., Am. J. Physiol. Renal Physiol., 292:F1761-F1770, 2007); a mouse model for asthma, suggested to be primarily through the S1P₁ receptor on the basis of work using the S1P₁ receptor agonist SEW2871 (Idzko et al., J Clin. Invest., 116:2935-2944, 2006); a mouse model for airway inflammation and induction of bronchial hyperresponsiveness (Sawicka et al., J. Immunol., 171; 6206-6214, 2003); a mouse model for atopic dermatitis (Kohno et al., Biol. Pharm. Bull., 27:1392-1396, 2004); a mouse model for ischemia-reperfusion injury (Kaudel et al., Transplant. Proc., 39:499-502, 2007); a mouse model for systemic lupus erythematosus (SLE) (Okazaki et al., J. Rheumatol., 29:707-716, 2002; Herzinger et al., Am. J. Clin. Dermatol., 8:329-336, 2007); rat models for rheumatoid arthritis (Matsuura et al., Int. J. Immunopharmacol., 22:323-331, 2000; Matsuura et al., Inflamm. Res., 49:404-410, 2000); a rat model for autoimmune uveitis (Kurose et al., Exp. Eye Res., 70:7-15, 2000); mouse models for type I diabetes (Fu et al., Transplantation, 73:1425-1430, 2002; Maki et al., Transplantation, 74:1684-1686, 2002; Yang et al., Clinical Immunology, 107:30-35, 2003; Maki et al., Transplantation, 79:1051-1055, 2005); mouse models for atherosclerosis (Nofer et al., Circulation, 115:501-508, 2007; Keul et al., Arterioscler. Thromb. Vasc. Biol., 27:607-613, 2007); a rat model for brain inflammatory reaction following traumatic brain injury (TBI) (Zhang et al., J Cell. Mol. Med., 11:307-314, 2007); and mouse models for graft coronary artery disease and graft-versus-host disease (GVHD) (Hwang et al., Circulation, 100:1322-1329, 1999; Taylor et al., Blood, 110:3480-3488, 2007). In vitro results suggest that FTY720 may have therapeutic efficacy for P-amyloid-related inflammatory diseases including Alzheimer's disease (Kaneider et al., FASEB J, 18:309-311, 2004). KRP-203, an S1P receptor agonist having agonist activity on the S1P₁ receptor, has been reported to have therapeutic efficacy in a rat model for autoimmune myocarditis (Ogawa et al., BBRC, 361:621-628, 2007). Using the S1P₁ receptor agonist SEW2871, it has been shown that agonism of endothelial S1P₁ receptors prevents proinflammatory monocyte/endothelial interactions in type I diabetic vascular endothelium (Whetzel et al., Circ. Res., 99:731-739, 2006) and protects the vasculature against TNFα-mediated monocyte/endothelial interactions (Bolick et al., Arterioscler. Thromb. Vasc. Biol., 25:976-981, 2005).

Additionally, FTY720 has been reported to have therapeutic efficacy in experimental autoimmune encephalomyelitis (EAE) in rats and mice, a model for human multiple sclerosis (Brinkmann et al., J. Biol. Chem., 277:21453-21457, 2002; Fujino et al., J Pharmacol. Exp. Ther., 305:70-77, 2003; Webb et al., J Neuroimmunol., 153:108-121, 2004; Rausch et al., J Magn. Reson. Imaging, 20:16-24, 2004; Kataoka et al., Cellular & Molecular Immunology, 2:439-448, 2005; Brinkmann et al., Pharmacology & Therapeutics, 115:84-105, 2007; Baumruker et al., Expert Opin. Investig. Drugs, 16:283-289, 2007; Balatoni et al., Brain Research Bulletin, 74:307-316, 2007). Furthermore, FTY720 has been found to have therapeutic efficacy for multiple sclerosis in clinical trials. In Phase II clinical trials for relapsing-remitting multiple sclerosis, FTY720 was found to reduce the number of lesions detected by magnetic resonance imaging (MRI) and clinical disease activity in individuals with multiple sclerosis (Kappos et al., N. Engl. J. Med., 355:1124-1140, 2006; Martini et al., Expert Opin. Investig. Drugs, 16:505-518, 2007; Zhang et al., Mini-Reviews in Medicinal Chemistry, 7:845-850, 2007; Brinkmann, Pharmacology & Therapeutics, 115:84-105, 2007). Phase III clinical studies with FTY720 in individuals with remitting-relapsing multiple sclerosis have been reported (Brinkmann, Pharmacology & Therapeutics, 115:84-105, 2007; Baumruker et al., Expert. Opin. Investig. Drugs, 16:283-289, 2007; Dev et al., Pharmacology and Therapeutics, 117:77-93, 2008).

FTY720 has also been reported to have anti-viral activity. Specific data has been presented in the lymphocytic choriomeningitis virus (LCMV) mouse model, wherein the mice were infected with either the Armstrong or the clone 13 strain of LCMV (Premenko-Lanier et al., Nature, 454, 894, 2008).

FTY720 has been reported to impair migration of dendritic cells infected with Francisella tularensis to the mediastinal lymph node, thereby reducing the bacterial colonization of it. Francisella tularensis is associated with tularemia, ulceroglandular infection, respiratory infection and a typhoidal disease (E. Bar-Haim et al., PLoS Pathogens, 4(11): e1000211, published 21 Nov. 2008; info:doi/10.1371/journal.ppat.1000211, 2008).

It has also been recently reported that a short-term high dose of FTY720 rapidly reduced ocular infiltrates in experimental autoimmune uveoretinitis. When given in the early stages of ocular inflammation, FTY720 rapidly prevented retinal damage. It was reported that FTY720 not only prevented infiltration of target organs, but also reduce existing infiltration (Raveney et al., Arch. Ophthalmol., 126(10), 1390, 2008).

It has been reported that treatment with FTY720 relieved ovariectomy-induced osteoporosis in mice by reducing the number of mature osteoclasts attached to the bone surface. The data provided evidence that S1P controlled the migratory behavior of osteoclast precursors, dynamically regulating bone mineral homeostasis (Ishii et al., Nature, advance online publication, 8 Feb. 2009, doi:10.1038/nature07713).

Agonism of the S1P₁ receptor has been implicated in enhancement of survival of oligodendrocyte progenitor cells. Survival of oligodendrocyte progenitor cells is a required component of the remyelination process. Remyelination of multiple sclerosis lesions is considered to promote recovery from clinical relapses (Miron et al., Ann. Neurol., 63:61-71, 2008; Coelho et al., J. Pharmacol. Exp. Ther., 323:626-635, 2007; Dev et al., Pharmacology and Therapeutics, 117:77-93, 2008). It also has been shown that the S1P₁ receptor plays a role in platelet-derived growth factor (PDGF)-induced oligodendrocyte progenitor cell mitogenesis (Jung et al., Glia, 55:1656-1667, 2007).

Agonism of the S1P₁ receptor has also been reported to mediate migration of neural stem cells toward injured areas of the central nervous system (CNS), including in a rat model of spinal cord injury (Kimura et al., Stem Cells, 25:115-124, 2007).

Agonism of the S1P₁ receptor has been implicated in the inhibition of keratinocyte proliferation (Sauer et al., J. Biol. Chem., 279:38471-38479, 2004), consistent with reports that S1P inhibits keratinocyte proliferation (Kim et al., Cell Signal, 16:89-95, 2004). The hyperproliferation of keratinocytes at the entrance to the hair follicle, which can then become blocked, and an associated inflammation are significant pathogenetic factors of acne (Koreck et al., Dermatology, 206:96-105, 2003; Webster, Cutis, 76(2 Suppl):4-7, 2005).

FTY720 has been reported to have therapeutic efficacy in inhibiting pathologic angiogenesis, such as that as may occur in tumor development. Inhibition of angiogenesis by FTY720 is thought to involve agonism of the S1P₁ receptor (Oo et al., J. Biol. Chem., 282; 9082-9089, 2007; Schmid et al., J. Cell Biochem., 101:259-270, 2007). FTY720 has been reported to have therapeutic efficacy for inhibiting primary and metastatic tumor growth in a mouse model of melanoma (LaMontagne et al., Cancer Res., 66:221-231, 2006). FTY720 has been reported to have therapeutic efficacy in a mouse model for metastatic hepatocellular carcinoma (Lee et al., Clin. Cancer Res., 11:84588466, 2005).

It has been reported that oral administration of FTY720 to mice potently blocked VEGF-induced vascular permeability, an important process associated with angiogenesis, inflammation, and pathological conditions such as sepsis, hypoxia, and solid tumor growth (T Sanchez et al., J. Biol. Chem., 278(47), 47281-47290, 2003).

Cyclosporin A and FK506 (calcineurin inhibitors) are drugs used to prevent rejection of transplanted organs. Although they are effective in delaying or suppressing transplant rejection, classical immunosuppressants such as cyclosporin A and FK506 are known to cause several undesirable side effects including nephrotoxicity, neurotoxicity, β-cell toxicity and gastrointestinal discomfort. There is an unmet need in organ transplantation for an immunosuppressant without these side effects which is effective as a monotherapy or in combination with a classical immunosuppressant for inhibiting migration of, e.g., alloantigen-reactive T-cells to the grafted tissue, thereby prolonging graft survival.

FTY720 has been shown to have therapeutic efficacy in transplant rejection both as a monotherapy and in synergistic combination with a classical immunosuppressant, including cyclosporin A, FK506, and RAD (an mTOR inhibitor). It has been shown that, unlike the classical immunosuppressants cyclosporin A, FK506 and RAD, FTY720 has efficacy for prolonging graft survival without inducing general immunosuppression, and this difference in drug action is believed to be relevant to the synergism observed for the combination (Brinkmann et al., Transplant Proc., 33:530-531, 2001; Brinkmann et al., Transplantation, 72:764-769, 2001).

Agonism of the S1P₁ receptor has been reported to have therapeutic efficacy for prolonging allograft survival in mouse and rat skin allograft models (Lima et al., Transplant Proc., 36:1015-1017, 2004; Yan et al., Bioorg. & Med. Chem. Lett., 16:3679-3683, 2006). FTY720 has been reported to have therapeutic efficacy for prolonging allograft survival in a rat cardiac allograft model (Suzuki et al., Transpl. Immunol., 4:252-255, 1996). FTY720 has been reported to act synergistically with cyclosporin A to prolong rat skin allograft survival (Yanagawa et al., J. Immunol., 160:5493-5499, 1998), to act synergistically with cyclosporin A and with FK506 to prolong rat cardiac allograft survival, and to act synergistically with cyclosporin A to prolong canine renal allograft survival and monkey renal allograft survival (Chiba et al., Cell Mol. Biol., 3:11-19, 2006). KRP-203, an S1P receptor agonist has been reported to have therapeutic efficacy for prolonging allograft survival in a rat skin allograft model and both as monotherapy and in synergistic combination with cyclosporin A in a rat cardiac allograft model (Shimizu et al., Circulation, 111:222-229, 2005). KRP-203 also has been reported to have therapeutic efficacy in combination with mycophenolate mofetil (MMF; a prodrug for which the active metabolite is mycophenolic acid, an inhibitor of purine biosynthesis) for prolonging allograft survival both in a rat renal allograft model and in a rat cardiac allograft model (Suzuki et al., J. Heart Lung Transplant, 25:302-209, 2006; Fujishiro et al., J. Heart Lung Transplant, 25:825-833, 2006). It has been reported that an agonist of the S1P₁ receptor, AUY954, in combination with a subtherapeutic dose of RAD001 (Certican/Everolimus, an mTOR inhibitor) can prolong rat cardiac allograft survival (Pan et al., Chemistry & Biology, 13:1227-1234, 2006). In a rat small bowel allograft model, FTY720 has been reported to act synergistically with cyclosporin A to prolong small bowel allograft survival (Sakagawa et al., Transpl. Immunol., 13:161-168, 2004). FTY720 has been reported to have therapeutic efficacy in a mouse islet graft model (Fu et al., Transplantation, 73:1425-1430, 2002; Liu et al., Microsurgery, 27:300-304; 2007) and in a study using human islet cells to evidence no detrimental effects on human islet function (Truong et al., American Journal of Transplantation, 7:2031-2038, 2007).

FTY720 has been reported to reduce the nociceptive behavior in the spared nerve injury model for neuropathic pain which does not depend on prostaglandin synthesis (O. Costu et al., Journal of Cellular and Molecular Medicine 12(3), 995-1004, 2008).

FTY720 has been reported to impair initiation of murine contact hypersensitivity (CHS). Adoptive transfer of immunized lymph node cells from mice treated with FTY720 during the sensitization phase was virtually incapable of inducing CHS response in recipients (D. Nakashima et al., J. Investigative Dermatology (128(12), 2833-2841, 2008).

It has been reported that prophylactic oral administration of FTY720 (1 mg/kg, three times a week), completely prevented the development of experimental autoimmune myasthenia gravis (EAMG) in C57BL/6 mice (T. Kohono et al., Biological & Pharmaceutical Bulletin, 28(4), 736-739, 2005).

In one embodiment, the present invention encompasses compounds which are agonists of the S1P₁ receptor having selectivity over the S1P₃ receptor. Using a combined chemical approach with S1P receptor null mice, Sanna et al. reported that sustained bradycardia was induced by nonselective S1P receptor immunosuppressive agonists in wild-type mice but was abolished in S1P₃−/− mice whereas an S1P₁-selective agonist did not produce bradycardia. Thus, suggesting that the S1P₃ receptor, and not the S1P₁ receptor, was responsible for bradycardia (Sanna et al., J. Biol. Chem., 279:13839-13848, 2004). Therefore, an S1P₁ receptor agonist selective over at least the S1P₃ receptor has advantages over current therapies by virtue of an enhanced therapeutic window, allowing better tolerability with higher dosing and thus improving efficacy as therapy. The present invention encompasses Compound 1 (and pharmaceutically acceptable salts, hydrates, and solvates thereof) which is an agonist of the S1P₁ receptor and has exhibited no or substantially no bradycardia in male Sprague-Dawley® rats (see WO2010/011316, Example 9).

A phase 1 study with Compound 1 was conducted with single dosing at 0.1 mg, 0.35 mg, 1 mg, 3 mg, and 5 mg. Compound 1 was administered as the L-arginine salt. Lower doses of 0.1 mg through 3 mg were well tolerated by subjects with only minor adverse events reported, the most common of which were headache and contact dermatitis. A dose-dependent reduction in heart rate was seen in all doses >0.35 mg, however, no adverse events related to bradycardia were reported at doses lower than the 5 mg dose. Dose limiting adverse events were observed at the dose of 5 mg, with 3 (50%) subjects experiencing 4 AEs of bradycardia with first or second degree atrioventricular (AV) block, which resulted in discontinuation of dose escalation. The maximum tolerated dose in the study was 3 mg. There were no deaths or serious adverse events in the study.

There were no other clinically significant safety issues with respect to vital signs, ECGs, pulmonary function tests, ophthalmoscopy, or clinical laboratory tests with the exception of expected pharmacological effects on peripheral blood lymphocyte counts. Dosing at the 3 and 5 mg induced a dose responsive decline in the absolute number of peripheral blood B cells, T cells, NK cells, and all T cell subsets except TEM cells. Total peripheral blood lymphocyte (PBL) counts were reduced by 2-4 hours after dosing, reaching a nadir by hour 8 which persisted for 24 hours with recovery to baseline over the next 4 days. PBL counts were reduced by ˜40% and ˜55% at the 3 mg and 5 mg dose levels. TEM cells do not express CCR7 and are able to recirculate independently of S1P receptor expression. These findings are therefore consistent with the anticipated pharmacodynamic effects of S1P receptor agonists in preclinical studies and in humans (Gergely et al., Br J Pharmacol 167(5):1035-1047, 2012; Brossard et al., Br J Clin Pharmacol 2013 Apr. 18. doi:10.1111/bcp.12129. [Epub ahead of print] PubMed PMID: 23594176, and Kovarik et al., J Clin Pharmacol 44(5):532-537, 2004.)

S1P₁ receptor agonists are useful to treat or prevent conditions where suppression of the immune system or agonism of the S1P₁ receptor is in order, such as diseases and disorders mediated by lymphocytes, transplant rejection, autoimmune diseases and disorders, inflammatory diseases and disorders, and conditions that have an underlying defect in vascular integrity or that relate to angiogenesis such as may be pathologic.

In one embodiment, the present invention encompasses compounds which are agonists of the S1P₁ receptor having good overall physical properties and biological activities and having an effectiveness that is substantially at least that of prior compounds with activity at the S1P₁ receptor.

S1P₁ receptor agonists are useful for treating or preventing conditions where suppression of the immune system or agonism of the S1P₁ receptor is in order, such as diseases and disorders mediated by lymphocytes, transplant rejection, autoimmune diseases and disorders, inflammatory diseases and disorders (e.g., acute and chronic inflammatory conditions), cancer, and conditions that have an underlying defect in vascular integrity or that are associated with angiogenesis such as may be pathologic (e.g., as may occur in inflammation, tumor development and atherosclerosis). Such conditions where suppression of the immune system or agonism of the S1P₁ receptor is in order include diseases and disorders mediated by lymphocytes; conditions that have an underlying defect in vascular integrity; autoimmune diseases and disorders; inflammatory diseases and disorders (e.g., acute and chronic inflammatory conditions); acute or chronic rejection of cells; tissue or solid organ grafts; arthritis, including psoriatic arthritis, and rheumatoid arthritis; diabetes, including type I diabetes; demyelinating disease, including multiple sclerosis; ischemia-reperfusion injury, including renal and cardiac ischemia-reperfusion injury; inflammatory skin disease, including psoriasis, atopic dermatitis, and acne; hyperproliferative skin disease, including acne; inflammatory bowel disease, including Crohn's disease, and ulcerative colitis; systemic lupus erythematosus; asthma; uveitis; myocarditis; allergy; atherosclerosis; brain inflammation, including Alzheimer's disease, and brain inflammatory reaction following traumatic brain injury; ankylosing spondylitis; central nervous system disease, including spinal cord injury, or cerebral infarction; pathologic angiogenesis, including as may occur in primary and metastatic tumor growth; rheumatoid arthritis; diabetic retinopathy, atherosclerosis; cancer; chronic pulmonary disease; acute lung injury; acute respiratory disease syndrome; sepsis; and the like. In addition, S1P₁ receptor agonists are useful for treating microbial infections, and viral infections or diseases.

In some embodiments, the sphingosine 1-phosphate subtype 1 (S1P₁) receptor-associated disorder is selected from: a disease or disorder mediated by lymphocytes, an autoimmune disease or disorder, an inflammatory disease or disorder, ankylosing spondylitis, biliary cirrhosis, cancer, psoriasis, psoriatic arthritis, rheumatoid arthritis, Crohn's disease, transplant rejection, multiple sclerosis, systemic lupus erythematosus, inflammatory bowel disease, ulcerative colitis, type I diabetes, hypertensive nephropathy, glomerulosclerosis, myocardial ischemia-reperfusion injury and acne.

In some embodiments, the S1P₁ receptor-associated disorder is a disease or disorder mediated by lymphocytes.

In some embodiments, the S1P₁ receptor-associated disorder is an autoimmune disease or disorder.

In some embodiments, the S1P₁ receptor-associated disorder is an inflammatory disease or disorder.

In some embodiments, the S1P₁ receptor-associated disorder is ankylosing spondylitis.

In some embodiments, the S1P₁ receptor-associated disorder is biliary cirrhosis.

In some embodiments, the S1P₁ receptor-associated disorder is primary biliary cholangitis.

In some embodiments, the S1P₁ receptor-associated disorder is cancer.

In some embodiments, the S1P₁ receptor-associated disorder is psoriasis.

In some embodiments, the S1P₁ receptor-associated disorder is erythema nodosum.

In some embodiments, the S1P₁ receptor-associated disorder is pyoderma gangrenosum.

In some embodiments, the S1P₁ receptor-associated disorder is psoriatic arthritis.

In some embodiments, the S1P₁ receptor-associated disorder is rheumatoid arthritis.

In some embodiments, the S1P₁ receptor-associated disorder is Crohn's disease.

In some embodiments, the S1P₁ receptor-associated disorder is transplant rejection.

In some embodiments, the S1P₁ receptor-associated disorder is multiple sclerosis.

In some embodiments, the S1P₁ receptor-associated disorder is systemic lupus erythematosus.

In some embodiments, the S1P₁ receptor-associated disorder is inflammatory bowel disease (IBD).

In some embodiments, the S1P₁ receptor-associated disorder is an active skin extra-intestinal manifestation of inflammatory bowel disease. In some embodiments, the S1P₁ receptor-associated disorder is an active skin extra-intestinal manifestation of ulcerative colitis. In some embodiments, the active skin extra-intestinal manifestation is psoriasis. In some embodiments, the active skin extra-intestinal manifestation is erythema nodosum. In some embodiments, the active skin extra-intestinal manifestation is pyoderma gangrenosum.

In some embodiments, the S1P₁ receptor-associated disorder is ulcerative colitis. In some embodiments, the S1P₁ receptor-associated disorder is moderately to severely active ulcerative colitis. In some embodiments, the S1P₁ receptor-associated disorder is moderately active ulcerative colitis. In some embodiments, the S1P₁ receptor-associated disorder is severely active ulcerative colitis. In some embodiments, the S1P₁ receptor-associated disorder is mildly to moderately active ulcerative colitis. In some embodiments, the S1P₁ receptor-associated disorder is mildly active ulcerative colitis.

In some embodiments, the S1P₁ receptor-associated disorder is type I diabetes.

In some embodiments, the S1P₁ receptor-associated disorder is hypertensive nephropathy.

In some embodiments, the S1P₁ receptor-associated disorder is glomerulosclerosis.

In some embodiments, the S1P1 receptor-associated disorder is myocardial ischemia-reperfusion injury.

In some embodiments, the S1P₁ receptor-associated disorder is acne.

In some embodiments, the S1P₁ receptor-associated disorder is autoimmune hepatitis.

In some embodiments, the standard dose is in an amount equivalent to 1 mg of Compound 1.

In some embodiments, the standard dose is in an amount equivalent to 2 mg of Compound 1.

In some embodiments, the standard dose is in an amount equivalent to 3 mg of Compound 1.

In some embodiments, the standard dose of Compound 1, or a pharmaceutically acceptable salt, hydrate, or solvate thereof is administered once daily to the individual.

In some embodiments, the Compound 1, or a pharmaceutically acceptable salt, hydrate, or solvate thereof, is administered orally.

In some embodiments, the Compound 1, or a pharmaceutically acceptable salt, hydrate, or solvate thereof, is formulated as a capsule or tablet suitable for oral administration.

In some embodiments, the Compound 1, or a pharmaceutically acceptable salt, hydrate, or solvate thereof, is selected from: Compound 1; a calcium salt of Compound 1; and an L-arginine salt of Compound 1. In some embodiments, the Compound 1, or a pharmaceutically acceptable salt, hydrate, or solvate thereof, is an L-arginine salt of Compound 1. In some embodiments, the Compound 1, or a pharmaceutically acceptable salt, hydrate, or solvate thereof, is an anhydrous, non-solvated crystalline form of an L-arginine salt of Compound 1. In some embodiments, the Compound 1, or a pharmaceutically acceptable salt, hydrate, or solvate thereof, is an anhydrous, non-solvated crystalline form of Compound 1.

In some embodiments, the individual also is administered a therapeutic dose of an oral 5-ASA compound. In some embodiments, the individual also is administered a stable dose of an oral 5-ASA compound.

In some embodiments, the individual also is administered a therapeutic dose of an oral corticosteroid therapy. In some embodiments, the individual also is administered a stable dose of an oral corticosteroid therapy. In some embodiments, the corticosteroid is prednisone, e.g., prednisone at a dose <10 mg/day or 20 mg/day, or an equivalent steroid. In some embodiments, the corticosteroid is budesonide, e.g., at a dose ≤9 mg/day, or an equivalent steroid.

In some embodiments, the individual also is administered a therapeutic dose of an immunosuppressive agent. In some embodiments, the individual also is administered a therapeutic dose of a thiopurine. In some embodiments, the individual also is administered a therapeutic dose of azathioprine. In some embodiments, the individual also is administered a therapeutic dose of 6-mercaptopurine. In some embodiments, the individual also is administered a therapeutic dose of thioguanine (also referred to as tioguanine or 6-thioguanine).

In some embodiments, the individual also is administered a therapeutic dose of a probiotic. In some embodiments, the individual also is administered a therapeutic dose of Culturelle. In some embodiments, the individual also is administered a therapeutic dose of Saccharomyces boulardii.

In some embodiments, the individual also is administered a therapeutic dose of an antidiarrheal. In some embodiments, the individual also is administered a therapeutic dose of loperamide. In some embodiments, the individual also is administered a therapeutic dose of diphenoxylate with atropine.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

Also provided are pharmaceutical compositions comprising a standard dose of Compound 1, or, a pharmaceutically acceptable salt, a hydrate or solvate thereof and, optionally, one or more pharmaceutically acceptable carriers. Also provided are pharmaceutical compositions comprising Compound 1, or, a pharmaceutically acceptable salt, a hydrate or solvate thereof, optionally, one or more pharmaceutically acceptable carriers. The carrier(s) must be “acceptable” in the sense of being compatible with the other ingredients of the formulation and not overly deleterious to the recipient thereof.

In some embodiments, Compound 1, or, a pharmaceutically acceptable salt, a hydrate or solvate thereof, is administered as a raw or pure chemical, for example as a powder in capsule formulation.

In some embodiments, Compound 1, or, a pharmaceutically acceptable salt, a hydrate or solvate thereof, is formulated as a pharmaceutical composition further comprising one or more pharmaceutically acceptable carriers.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary. Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Further embodiments include the embodiments disclosed in the following Examples, which is not to be construed as limiting in any way.

### EXAMPLES

### Example 1

Formulations composed of immediate-release, hard gelatin capsules containing an L-arginine salt of Compound 1 were prepared as shown in Table 1.

TABLE 1

| Formulation | 0.1 mg | 0.35 mg | 0.5 mg | 1 mg | 2 mg |
|---|---|---|---|---|---|
| L-arginine salt of Com- | 0.14 | 0.48 | 0.69 | 1.38 | 2.76 |
| pound 1 (mg/capsule) | | | | | |
| Empty capsule weight (mg)* | 38.0 | 61.0 | 61.0 | 61.0 | 61.0 |
| Total capsule target | 38.14 | 61.48 | 61.69 | 62.38 | 63.76 |
| weight (mg)** | | | | | |

*Approximate weight. Based on capsule specification
**Theoretical total weight calculated by combining fill and empty capsule weights together

### Example 2

Formulations composed of immediate-release tablets containing an L-arginine salt of Compound 1 were prepared as shown in Table 2.

TABLE 2

| Tablet Strength | 0.5 mg | 1 mg | 2 mg | 3 mg |
|---|---|---|---|---|
| L-Arg Salt of Compound 1 | 0.69 | 1.381 | 2.762 | 4.143 |
| Mannitol Pearlitol ® 100 SD | 54.81 | 54.119 | 52.738 | 51.357 |
| Microcrystalline | 40 | 40 | 40 | 40 |
| cellulose-Avicel ® | | | | |
| Sodium Starch | 4 | 4 | 4 | 4 |
| Glycolate-Explotab ® | | | | |
| Magnesium Stearate | 0.5 | 0.5 | 0.5 | 0.5 |
| Opadry ® II Blue | 4 | 4 | 4 | 4 |
| Total tablet target weight | 104 | 104 | 104 | 104 |

### Example 3

### In Vitro Evaluation of Compound 1 as an Inhibitor of Cytochrome P450 (CYP) and UDP-Glucuronosyltransferase (UGT) Enzymes in Human Liver Microsomes

Compound 1 was evaluated for potential inhibition of the cytochrome P450 (CYP) enzymes CYP1A2, CYP2B6, CYP2C8, CYP2C9, CYP2C19, CYP2D6, CYP3A4/5 (using two different marker substrates) and UDP-glucuronosyltransferase (UGT) enzymes UGT1A1, UGT1A3, UGT1A4, UGT1A6, UGT1A9, UGT2B7 and UGT2B17 in human liver microsomes, with the aim of ascertaining the potential of Compound 1 to inhibit the metabolism of concomitantly administered drugs.

To evaluate Compound 1 as a direct and metabolism-dependent inhibitor of CYP activity and as a direct inhibitor of UGT activity, human liver microsomes from a pool of 200 individuals were incubated with marker substrates in the presence or absence of Compound 1 at concentrations of 0, 1 or 10 M. For metabolism-dependent inhibition of CYP enzymes, Compound 1 was preincubated with human liver microsomes for 30 minutes with an NADPH-regenerating system, prior to the incubation with the marker substrates. Known metabolism-dependent and/or direct inhibitors of CYP and UGT enzymes were included as positive controls, as applicable.

After 120 minutes of incubation with rCYP enzymes, Compound 1 loss was observed to the greatest extent with rCYP2C8 (up to 75%) and ranged from 12 to 36% loss with rCYP2C9, rCYP2C19 and rCYP3A4. In the HLMs without chemical inhibitors, overall loss of Compound 1 was negligible (0 to 13.0%). A selective CYP2C8 inhibitor (gemfibrozil) inhibited, by 85%, 47%, and 60%, the limited conversion of Compound 1 to two oxidative metabolites and a ketone metabolite, respectively. The limited conversion of Compound 1 to the second oxidative metabolite was also inhibited (72%) by a CYP2C9 inhibitor (tienilic acid).

Direct inhibition of CYP2C8 activity was 22% at 1 μM and ˜100% at 10 μM in the presence of Compound 1. Little or no direct inhibition of CYP1A2, CYP2B6, CYP2C9, CYP2C19, CYP2D6 or CYP3A4/5 activities was observed in the presence of Compound 1 concentrations up to 10 μM. In addition, little or no evidence of metabolism-dependent inhibition of CYPs was seen.

Treatment of cultured human hepatocytes with Compound 1 was found to have no induction potential on CYP1A2 mRNA. Additionally, no induction potential for CYP2B6 and CYP3A4 was seen, based on induction criteria being a >2-fold increase in mRNA and ≥20% of the mRNA increase seen with positive control.

Compound 1 directly inhibited UGT1A1 and UGT1A6 activities by up to 28% and 48%, respectively, at the highest concentration tested (10 μM). There was no evidence of direct inhibition of UGT1A3, UGT1A4, UGT1A9, UGT2B7 or UGT2B17 as less than 8% inhibition was observed in the presence of Compound 1 concentrations up to 10 M.

Based on the results from two approaches to CYP reaction phenotyping, CYP2C8 and CYP2C9 were established to play major roles in conversion of Compound 1 to oxidative metabolites, and CYP2C8 also plays a major role in formation of a ketone metabolite. However, the overall conversion of Compound 1 to metabolites was negligible in HLMs and there was little difference in the loss of Compound 1 seen in the presence and absence of direct-acting and metabolism-dependent selective CYP inhibitors. Direct inhibition of CYP2C8 activity was 22% at 1 μM and ˜100% at 10 μM in the presence of Compound 1. However, there was little or no direct inhibition observed for any other CYPs with Compound 1. Compound 1 directly inhibited UGT1A1 and UGT1A6 activities by up to 28% and 48%, respectively, at the highest concentration tested (10 μM), but there was no evidence of direct inhibition of UGT1A3, UGT1A4, UGT1A9, UGT2B7 or UGT2B17. Compound 1 (up to 10 μM) was not a potential inducer of CYP1A2, CYP2B6, and CYP3A4/5.

### Example 4

Compound 1 was evaluated as a potential substrate and/or inhibitor of human ABC transporters P-gp, BCRP and BSEP or human SLC transporters OATP1B1, OATP1B3, OAT1, OAT3, OCT1, OCT2, MATE1 and MATE2-K. P-gp and BCRP are expressed on the apical membrane of a number of tissues. P-gp and BCRP are expressed in the luminal membrane of enterocytes, endothelial cells in the brain, the brush border membrane of renal proximal tubules and the canalicular membrane of hepatocytes where they limit the intestinal absorption, blood-brain barrier penetration and facilitate excretion into the bile and urine. BSEP is mainly expressed in the canalicular membrane of hepatocytes where it facilitates excretion into the bile. OATP1B1, OATP1B3 and OCT1 are expressed on the sinusoidal membrane of hepatocytes and facilitate the accumulation of endogenous and xenobiotic compounds into hepatocytes for further metabolism or excretion into the bile. OAT1, OAT3 and OCT2 are expressed on the basolateral membrane of renal proximal tubules and facilitate the accumulation of compounds into the proximal tubule for further excretion in the urine. MATE1 and MATE2-K (multidrug and toxin extrusion proteins) are primarily expressed on the luminal (apical) membrane of the proximal tubular cells and thought to play a role in the excretion of cations and zwitterions into urine. MATE1 is also expressed in the liver on the canalicular membrane of hepatocytes and mediates the biliary excretion of cationic drugs. MATE1 and MATE2-K may function in cooperation with OCT transporters expressed on the canalicular membranes of hepatocytes and the basolateral membranes of proximal tubules to mediate excretion. Compounds that are substrates or inhibitors of the transporters may be victims or perpetrators in drug-drug interactions.

Madin-Darby canine kidney cells (MDCKII), over-expressing human permeability-glycoprotein (P-gp) and breast cancer resistance protein (BCRP), were used to evaluate Compound 1 as a substrate of P-gp and BCRP, and as an inhibitor of BCRP. A polarized cell line, derived from a human colon carcinoma (Caco 2) cells, was used to evaluate Compound 1 as an inhibitor of P-gp. Membrane vesicles expressing bile salt export pump (BSEP) were used in a vesicular transport assay to evaluate Compound 1 as an inhibitor of BSEP. Human embryonic kidney cells (HEK293) transfected with vectors containing human transporter cDNA for organic anion-transporting polypeptide 1B1 (OATP1B1), organic anion-transporting polypeptide 1B3 (OATP1B3), organic anion transporter 1 (OAT1), organic anion transporter 3 (OAT3), organic cation transporter 1 (OCT1), organic cation transporter 2 (OCT2) and control cells (HEK293 cells transfected with only vector) were used in experiments to evaluate Compound 1 as a substrate and an inhibitor of OATP1B1, OATP1B3, OAT1, OAT3, OCT1 and OCT2. HEK293 cells transfected with vectors containing renal multidrug and toxin extrusion transporters 1 and 2-K (MATE1, MATE2-K) were used for evaluating Compound 1 as an inhibitor of MATE1 and MATE2-K. Known substrates and inhibitors of ABC and SLC transporters were included as positive controls in all experiments.

There was no P-gp inhibition at 10 M. In the presence of 100 μM Compound 1, the efflux ratio of digoxin (10 μM) across Caco-2 cells was reduced by ˜50% indicating that Compound 1 is an inhibitor of P-gp with an IC50 value of ˜100 M.

In the presence of Compound 1 (10 and 100 μM), the corrected efflux ratio of prazosin (1 μM) across MDCKII-BCRP cells was reduced by more than 50%; however, inhibition potential of Compound 1 could not be determined since post-assay TEER values were below acceptance criteria and lucifer yellow Papp values were above acceptable ranges at both 10 and 100 M. To determine an IC50, a second experiment was performed with seven concentrations of Compound 1 ranging from 0.03 to 30 M. The resulting IC50 value was 35.7 M.

In the presence of Compound 1 (1 and 10 μM), the ATP-dependent uptake of [3H]-taurocholic acid in BSEP-expressing vesicles was reduced by less than 50% indicating that Compound 1 is not an inhibitor of BSEP at the concentrations evaluated.

There was no OATP1B1 inhibition at 1 μM Compound 1. In the presence of 10 μM Compound 1, the uptake rate of [3H]-estradiol-170-glucuronide (50 nM) into OATP1B1-expressing cells was reduced by ˜50% indicating Compound 1 is an inhibitor of OATP1B1 with an IC50 value of ˜10 M.

In the presence of Compound 1 (1 and 10 μM), the uptake rate of [3H]-estradiol-170-glucuronide (50 nM) into OATP1B3-expressing cells was reduced by less than 50% indicating Compound 1 is not an inhibitor of OATP1B3 at the concentrations evaluated (IC50>10 μM).

In the presence of Compound 1 (1 and 10 μM), the uptake rate of [3H]-p-aminohippurate (1 μM) into OAT1-expressing cells was reduced by less than 50% indicating Compound 1 is not an inhibitor of OAT1 at the concentrations evaluated (IC50>10 μM).

In the presence of Compound 1 (1 and 10 μM), the uptake rate of [3H]-estrone-3-sulfate (50 nM) into OAT3-expressing cells was reduced by less than 50% indicating Compound 1 is not an inhibitor at the concentrations evaluated (IC50>10 μM).

In the presence of Compound 1 (1 and 10 μM), the uptake rate of [14C]-tetraethylammonium bromide (5 μM) into OCT1-expressing cells was reduced by less than 50% indicating Compound 1 is not an inhibitor at the concentrations evaluated (IC50>10 μM).

In the presence of Compound 1 (1 and 10 μM), the uptake rate of [14C]-metformin (10 μM) into OCT2-expressing cells was not reduced indicating Compound 1 is not an inhibitor of OCT2 at the concentrations evaluated (IC50>10 μM).

The efflux ratio of Compound 1 (1 μM) across MDCKII-P-gp cells was 1.12 and increased to 3.08 in the presence of the P-gp inhibitor valspodar (10 μM). The efflux ratio of Compound 1 (10 μM) across MDCKII-P-gp cells was 3.17 and did not reduce in the presence of valspodar. These results indicate Compound 1 is not a substrate of P-gp since the efflux ratio of Compound 1 was not reduced in the presence of inhibitor. It should be noted that Compound 1 recovery was low ranging from 18 to 60% and is likely due to non-specific binding to the plate.

The efflux ratio of Compound 1 across MDCKII-BCRP cells was less than two at 1 M, and more than two at 10 μM in the absence and presence of the BCRP inhibitor Ko143 (1 μM). Although the efflux ratio of Compound 1 at 10 μM was 7.36 and was reduced to 3.17 in the presence of Ko143 (1 μM), the efflux ratio data of Compound 1 at 1 μM indicate Compound 1 is not a substrate of BCRP. For consideration as a substrate of the BCRP transporter, the efflux ratio of Compound 1 would be appreciably higher at lower concentration (1 μM) than at higher concentration (10 μM), and the consequent inhibition effect would likely be higher at a lower concentration (1 μM). It should be noted that Compound 1 recovery was low ranging from 27 to 48% and is likely due to non-specific binding to the plate.

The uptake ratio of Compound 1 (1 and 10 μM) into OATP1B1-expressing cells was less than two in the absence and presence of the OATP1B1 inhibitor rifampin. These results show that Compound 1 is not a substrate of OATP1B1.

The uptake ratio of Compound 1 (1 and 10 μM) into OATP1B3-expressing cells was less than two in the absence and presence of the OATP1B3 inhibitor rifampin. These results show that Compound 1 is not a substrate of OATP1B3.

The uptake ratio of Compound 1 (1 and 10 μM) into OAT1-expressing cells was less than two in the absence and presence of the OAT1 inhibitor probenecid (100 μM). These results show that Compound 1 is not a substrate of OAT1.

The uptake ratio of Compound 1 (1 and 10 μM) into OAT3-expressing cells was below two in the absence and presence of the OAT3 inhibitor probenecid (100 μM). These results show that Compound 1 is not a substrate of OAT3.

The uptake ratio of Compound 1 (1 and 10 μM) into OCT1-expressing cells was below two in the absence and presence of the OCT1 inhibitor quinidine (100 μM). These results show that Compound 1 is not a substrate of OCT1.

The uptake ratio of Compound 1 (1 and 10 μM) into OCT2-expressing cells was below two in the absence and presence of the OCT2 inhibitor quinidine (300 μM). These results show that Compound 1 is not a substrate of OCT2.

TABLE 3
In Vitro Characterization of Compound 1 as a
Potential Substrate for Human ABC and SLC Transporters

| | | | Potential substrate |
|---|---|---|---|
| | | | (uptake or |
| | | | efflux ratio ≥2 |
| | | Compound 1 | and reduced in |
| Transporter | Test system | concentrations | presence of inhibitor) |
| P-gp | MDCKII-P-gp cells | 1 and 10 μM | No |
| BCRP | MDCKII-BCRP cells | | No |
| OATP1B1 | HEK293 cells | | No |
| OATP1B3 | | | No |
| OAT1 | | | No |
| OAT3 | | | No |
| OCT1 | | | No |
| OCT2 | | | No |

TABLE 4
In Vitro Characterization of Compound 1 as a
Potential Inhibitor of Human ABC and SLC Transporters

| | | | Compound 1 | IC₅₀ |
|---|---|---|---|---|
| Transporter | Test system | Substrate | concentrations | (μM) |
| P-gp | Caco-2 cells | Digoxin (10 μM) | 10 and 100 μM | ~100 ^{a} |
| BCRP | MDCKII-BCRP cells | Prazosin (1 μM) | 0.03 to 30 μM | 35.7 |
| BSEP | Vesicles | [³H]-Taurocholic acid (0.4 μM) | 1 and 10 μM | >10^{a} |
| OATP1B1 | HEK293 cells | [³H]-Estradiol-17β-glucuronide | | ~10^{a} |
| OATP1B3 | | (50 nM) | | >10^{a} |
| OAT1 | | [³H]-p-Aminohippurate (1 μM) | | >10^{a} |
| OAT3 | | [³H]-Estrone-3-sulfate (50 nM) | | >10^{a} |
| OCT1 | | [¹⁴C]-Tetraethylammonium | | >10^{a} |
| | | bromide (5 μM) | | |
| OCT2 | | [¹⁴C]-Metformin (10 μM) | | >10^{a} |
| MATE1 | | | | >10^{a} |
| MATE2-K | | | | >10^{a} |

^{a} Two test article concentrations were evaluated and <50% inhibition was observed

Overall, the results of this study showed the following:

Compound 1 was not a substrate of P-gp, BCRP, OATP1B1, OATP1B3, OAT1, OAT3, OCT1 and OCT2 transporters under the conditions evaluated. Compound 1 inhibited P-gp, BCRP and OATP1B1 with IC₅₀ values of ˜100, 35.7, and ˜10 μM, respectively. Compound 1 (up to 10 μM) caused less than 50% inhibition of all the other transporters examined (BSEP, OATP1B3, OAT1, OAT3, OCT1, OCT2, MATE1 and MATE2-K).

Compound 1 inhibited P-gp, BCRP and OATP1B1 with an IC50 values of ˜100, 35.7 and ˜10 M, respectively.

Compound 1 (up to 10 μM) caused less than 50% inhibition of the other transporters examined (BSEP, OATP1B3, OAT1, OAT3, OCT1, OCT2, MATE1 and MATE2-K). Compound 1 was found to have no inhibition potential with an IC50>10 μM on BSEP, OATP1B3, OAT1, OAT3, OCT1, OCT2, MATE1 and MATE2-K transporters.

### Example 5

Compound 1 was evaluated to assess the single dose relative oral bioavailability of 2 mg tablet and capsule formulations in the fasted state, determine the effect of food on the pharmacokinetics of the 2 mg tablet, assess potential gender differences in Compound 1 pharmacokinetics, and evaluate safety and tolerability in healthy adult subjects.

A randomized, single-dose, open-label, three-period, cross-over, phase 1 study was conducted in healthy adult subjects. A total of 14 subjects (7 males; 7 females) were randomized 1:1 into two groups, Sequence 1 and Sequence 2. Under fasted conditions in the first treatment period, the Sequence 1 group received a single 2 mg dose of the hard gelatin capsule formulation of Compound 1 (Treatment A), and the Sequence 2 group received a single 2 mg dose of the tablet formulation of Compound 1 (Treatment B). After a 7-day washout, the two groups crossed over in the second treatment period to receive the alternate treatment under fasted conditions. Following another 7-day washout, all subjects received Treatment C (a single 2 mg dose of the tablet formulation of Compound 1 under fed conditions; i.e., FDA-standard high-fat high-calorie meal) in the third treatment period. Blood samples for determination of plasma concentrations of Compound 1 were collected at prespecified time points up to 120 hours post dose. Plasma samples were analyzed for Compound 1 using a validated LC/MS/MS assay. The Compound 1 plasma concentration-time data were analyzed by noncompartmental methods in Phoenix™ WinNonlin® (Version 6.3, Pharsight Corporation) to determine plasma pharmacokinetic parameters including peak concentration (Cₘₐₓ), time to peak concentration (Tₘₐₓ), and area under the concentration-time curve from time-zero to the time of the last quantifiable concentration (AUC₀₋₄) or to infinity (AUC_{inf}). Consistent with the two one-sided test for bioequivalence, analysis of variance (ANOVA) was performed on In-transformed AUC₀₋₄, AUC_{mf} and Cₘₐₓ values to determine geometric mean ratios (Test/Reference treatments; see Tables 3 and 4 for treatment designations) and their associated 90% confidence intervals to assess relative bioavailability and food effects. Pharmacokinetic results were also stratified by gender to evaluate any potential differences between males and females. Safety and tolerability were evaluated using physical examination, ophthalmological examination, neurological and progressive multifocal leukoencephalopathy (PML) examination, vital sign measurements (supine blood pressure, heart rate, temperature, and respiratory rate), clinical laboratory evaluations, electrocardiograms (ECGs), telemetry monitoring, tuberculosis (TB) screening, pulmonary function testing (PFT, using spirometry), and reported or observed adverse events (AEs).

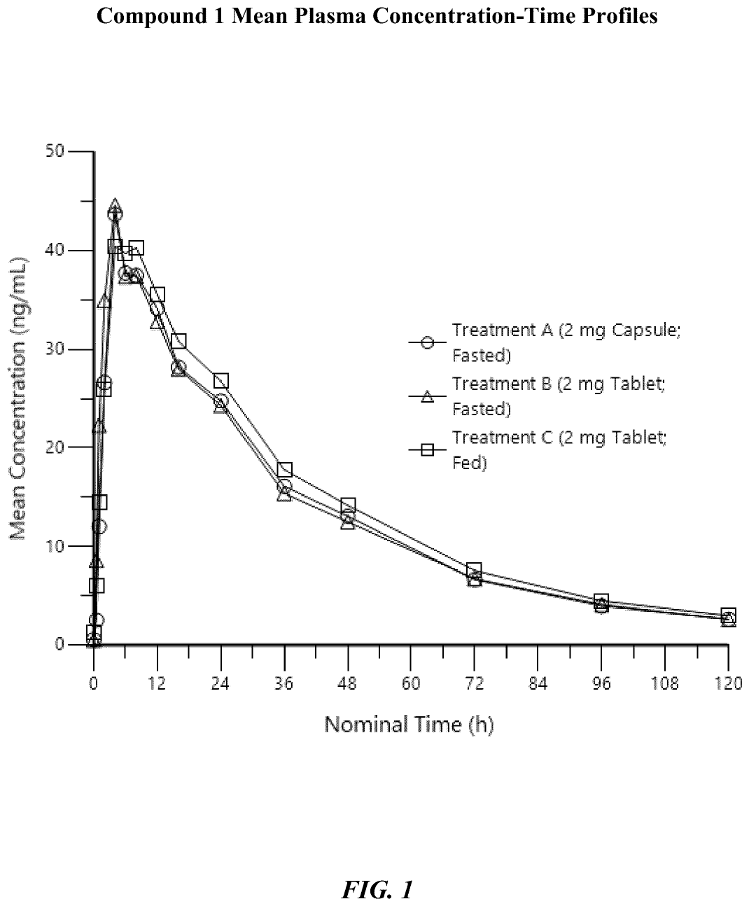

No significant differences between Compound 1 mean plasma concentration-time profiles were seen for the tablet and capsule formulations under fasted conditions (FIG. 1). The 90% confidence intervals for the geometric mean ratios of Compound 1 peak (Cₘₐₓ) and total (AUC) plasma exposure measures, comparing tablet versus capsule formulations, were within the accepted 80% to 125% range for establishing bioequivalence (Table 5).

No significant differences between Compound 1 mean plasma concentration-time profiles were seen for the tablet formulation administered under fed versus fasted conditions (FIG. 1). The 90% confidence intervals for the geometric mean ratios of Compound 1 peak (Cₘₐₓ) and total (AUC) plasma exposure measures, comparing the tablet under fed versus fasted conditions, were within the accepted 80% to 125% range for establishing no food effect.

Compound 1 mean plasma concentrations and exposure parameters were only moderately higher in females compared to males across treatments.

A total of eight AEs was reported by three subjects over the course of the study and consistent with what was seen previously in healthy volunteer studies. All AEs were considered treatment emergent, with only three AEs considered related to the administration of study medication. There were no serious AEs (SAEs) or AEs that led to subject discontinuation. No clinically significant abnormalities in ECGs or physical exams were observed. There were no AEs related to clinically significant out-of-range vital signs.

Compound 1 capsule and tablet formulations were found to be bioequivalent (interchangeable). No significant differences in Compound 1 exposure for the tablet formulation administered under fasted and fed conditions were demonstrated and thus Compound 1 can be taken without regard to meals. Observed modest gender differences in PK exposure measures were considered not likely clinically meaningful. Overall, the investigational product was well-tolerated in healthy subjects when administered as a 2 mg single dose under both fed and fasted conditions.

TABLE 5
Statistical Analysis Comparing Systemic Exposure of
2 mg Tablet under Fasted Conditions (Treatment B; Test)
to 2 mg Capsule under Fasted Conditions (Treatment A; Reference)

| PK Exposure | Geometric Mean^{a} | | GMR (%)^{b} | 90% CI^{c} | |
|---|---|---|---|---|---|
| Parameter | Test | Ref | (Test/Ref) | Lower | Upper |
| Cₘₐₓ | 43.49 | 44.26 | 98.25 | 91.42 | 105.59 |
| AUC₀₋₁₂₀ | 1551.17 | 1565.99 | 99.05 | 94.91 | 103.38 |
| AUC₀₋ₜ | 1551.2 | 1566.1 | 99.05 | 94.90 | 103.37 |
| AUC_{inf} | 1658.0 | 1703.8 | 97.31 | 92.82 | 102.02 |

^{a}Geometric Mean for 2 mg tablet under Fasted Conditions (Test) and 2 mg capsule under fasted conditions (Ref) based on Least Squares Mean of log-transformed parameter values; units are ng/ml and h · ng/ml for Cₘₐₓ and AUCs, respectively.
^{b}Geometric Mean Ratio (%) = Geometric Mean (Test)/Geometric Mean (Ref)
^{c}90% Confidence Interval

TABLE 6
Statistical Analysis Comparing Systemic Exposure of
2 mg Tablet under Fed Conditions (Treatment C; Test)
and Fasted Conditions (Treatment B; Reference)

| PK Exposure | Geometric Mean^{a} | | GMR (%)^{b} | 90% CI^{c} | |
|---|---|---|---|---|---|
| Parameter | Test | Ref | (Test/Ref) | Lower | Upper |
| Cₘₐₓ | 44.35 | 43.49 | 101.97 | 95.23 | 109.20 |
| AUC₀₋ₜ | 1691.2 | 1551.2 | 109.03 | 104.35 | 113.91 |
| AUC_{inf} | 1823.6 | 1658.0 | 109.99 | 104.88 | 115.35 |

^{a}Geometric Mean for 2 mg tablet under fed conditions (Test) and 2 mg tablet under fasted conditions (Ref) based on Least Squares Mean of log-transformed parameter values; units are ng/ml and h · ng/mL for Cₘₐₓ and AUCs, respectively.
^{b}Geometric Mean Ratio (%) = Geometric Mean (Test)/Geometric Mean (Ref)
^{c}90% Confidence Interval

### Example 6

Compound 1 (1 mg, 2 mg) is evaluated in an open-label, three-treatment, randomized, fixed sequence study to assess plasma pharmacokinetics (PK), pharmacodynamics (PD), safety, and tolerability in the presence and absence of dosing of fluconazole (a moderate inhibitor of CYP2C9), gemfibrozil (a strong inhibitor of CYP2C8), or rifampin (a moderate inducer of both CYP2C8 and CYP2C9) in healthy adult subjects.

Based on results from completed in vitro metabolism studies and a human mass balance clinical study, Compound 1 appears to be extensively metabolized (>25% overall) by oxidative (CYP2C8 and CYP2C9) and conjugation (UGT1A7, minor extent UGT1A1 and UGT1A4) pathways. Compound 1 is eliminated from the systemic circulation mainly due to metabolism and biliary/fecal excretion. Potential changes in Compound 1 pharmacokinetics are evaluated in the presence and absence of co-administered drugs that can impact one or more of these metabolism/elimination pathways-fluconazole or gemfibrozil due to the significant CYP inhibition effects, and rifampin due to the significant CYP induction effects.

The study entails one of three treatments: 1) Compound 1 (1 mg) alone and in the presence of steady state fluconazole (400 mg loading dose followed by daily oral dose 200 mg); 2) Compound 1 (1 mg) alone and in the presence of steady state gemfibrozil (600 mg BID oral dose); and 3) Compound 1 (2 mg) alone and in the presence of steady state rifampin (600 mg daily oral dose).

Each treatment consists of two periods: period 1 (administration of a single oral dose of Compound 1 alone) and period 2 (administration of Compound 1 in the presence of an inhibitor or inducer drug). Each period will be separated by 7-day washout period.

In period 1 on day 1, following an overnight fast, subjects (n=16 per treatment group) will receive a single oral dose of Compound 1 (1 mg for treatments A and B; 2 mg for treatment C) and single dose PK will be assessed over the next 7 days.

In period 2, all 16 subjects from each treatment group will be treated as follows:

Treatment A: On day 8, subjects will be administered a single dose of fluconazole (400 mg) followed by once daily dosing of fluconazole (200 mg) up to day 23. On day 12, following an overnight fast of at least 8 hours, a single dose of Compound 1 (1 mg) will be administered 30 min following daily dose of fluconazole.

Treatment B: On day 8, subjects will be administered twice daily gemfibrozil (600 mg) up to day 23. On day 12, following an overnight fast of at least 8 hours, a single dose of Compound 1 (1 mg) will be administered 30 minutes following daily dose of gemfibrozil.

Treatment C: On day 8, subjects will be administered once daily rifampin (600 mg) up to day 23. On day 15, following an overnight fast of at least 8 hours, a single dose of Compound 1 (2 mg) will be administered 30 min following daily dose of rifampin.

PK and PD profiles of Compound 1 are assessed after dose administration on day 1 (Compound 1 alone) and day 12/15 (in the presence of steady-state CYP2C8/CYP2C9 inhibitor or inducer). Measured PK parameters will include the following assessed using an analysis of covariance model for each comparison (i.e., Compound 1 alone versus the CYP2C8/CYP2C9 inhibitor or inducer):
Cmax: maximum concentration determined directly from the concentration time profile
tmax: time to reach maximum plasma concentration following drug administration determined directly from the concentration-time profile
t½: terminal elimination half-life calculated as: In2/λz
λz: terminal elimination rate constant determined by selection of at least 3 data points on the terminal phase of the concentration-time curve
AUC0-24: area under the concentration-time curve (AUC) zero to 24 hour calculated using the linear-log trapezoidal rule
AUC0-168: area under the concentration-time curve (AUC) zero to 168 hour calculated using the linear-log trapezoidal rule
AUC0-t
AUClast: AUC from time zero to the time of the last quantifiable concentration (tlast) calculated using the linear-log trapezoidal rule
AUC_{0-∞}: AUC from time zero to the infinity calculated using the linear-log trapezoidal rule
CL/F: total body clearance after oral administration
Vz/F: apparent volume of distribution after oral administration based on the terminal phase
MR: metabolic ratio calculated as AUC0-168(metabolite)/AUC0-168(parent)

Primary analyses evaluate the PK of Compound 1 and potential major metabolites to assess the extent of drug-drug interaction.

Other uses of the disclosed methods will become apparent to those in the art based upon, inter alia, a review of this patent document.

1. A method of treating an individual with a sphingosine 1-phosphate subtype 1 (S1P₁) receptor-associated disorder comprising:
administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof,
wherein the dosage form is administered under fed conditions,
wherein the therapeutically effective amount is equivalent to 2.0 mg of Compound 1,
wherein the pharmaceutical dosage form has a mean fed/fasted ratio of the area under the plasma concentration versus time curve of from about 0.8 to about 1.25 and a mean fed/fasted ratio of the maximum plasma concentration (Cmax) from about 0.8 to about 1.25,
wherein said administering results in no severe adverse event related to heart rate reduction or heart block in the individual.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. The method of claim 1, wherein the method is non-gender specific.

26. (canceled)

27. (canceled)

28. (canceled)

29. (canceled)

30. The method of claim 1, further comprising monitoring the individual for an active infection.

31. The method of claim 30, further comprising discontinuing administration if the individual develops an active infection.

32. The method of claim 1, wherein treating comprises inducing and/or maintaining clinical response; improving endoscopic appearance of the mucosa; and/or inducing and/or maintaining clinical remission.

33. The method of claim 1, wherein the Compound 1 is administered without titration.

34. The method of claim 1, wherein said administering results in no serious adverse events.

35. (canceled)

36. The method of claim 1, wherein the S1P₁ receptor-associated disorder is inflammatory bowel disease.

37. The method of claim 36, wherein the inflammatory bowel disease is ulcerative colitis.

38. The method of claim 37, wherein the inflammatory bowel disease is moderately to severely active ulcerative colitis.

39. The method of claim 36, wherein the inflammatory bowel disease is Crohn's disease.

40. The method of claim 36, wherein prior to said administering the individual has a 3-component Mayo Clinic Score of at least 6.

41. The method of claim 36, wherein said administering results in an improvement of the individual's 3-component Mayo Clinic Score.

42. The method of claim 36, wherein said administering results in an improvement of the individual's 2-component Mayo Clinic Score.

43. The method of claim 36, wherein said administering results in an improvement of the individual's Total Mayo Clinic Score.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. The method of claim 1, wherein the Compound 1, or a pharmaceutically acceptable salt, hydrate, or solvate thereof, is an L-arginine salt of Compound 1.

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. The method of claim 1, wherein Compound 1 is administered without causing more than 10 bpm of decrease in heart rate on first day of treatment.
